(19) United States
(12) Patent Application Publication
(10) Pub. No.: US 2009/0053813 A1
(43) Pub. Date: Feb. 26, 2009

(54) MULTIPLEXED ELECTROPORATION APPARATUS

(76) Inventor: DAVID MARK EVANS, NORTH POTOMAC, MD (US)

Correspondence Address: Saul Ewing LLP (Baltimore);Attn: Patent Docket Clerk, Lockwood Place, 500 East Pratt Street, Suite 900, Baltimore, MD, 21202, US

(21) Appl. No.: 12/196,707

(22) Filed: Aug. 22, 2008

Related U.S. Application Data
(60) Provisional application No. 60/965,942, filed on Aug. 24, 2007.

(51) Int. Cl. C12N 15/87; C12M 1/42; C40B 60/00; C12N 13/00

(52) U.S. Cl. 435/461; 435/285.2; 435/173.6; 435/471; 506/33

(57) ABSTRACT

Described herein are platforms and consumables for performing gene/siRNA/protein/peptide delivery screens in high throughput, including an automation-compatible transfection methodology that can be used to gain entry of oligonucleotides, proteins, peptides and other non-permeable molecules (e.g., some small organic compounds) into cells. Electrical stimulation is provided to cells, e.g., neurons or myocytes, in culture through application of pulsed fields directly to the cells in culture. The plate design is used in conjunction with a custom-designed circuit that allows the user to select pulse type, duration, etc.

### CROSS REFERENCE TO RELATED APPLICATIONS

This application claims the priority of copending U.S. provisional patent application No. 60/965,942, filed on Aug. 24, 2007, the entire contents of which are expressly incorporated herein by reference.

### FIELD

This application describes, among other things, systems and methods for automating transfection of oligonucleotides into cells, including an improved electroporation system and methods of use thereof.

### BACKGROUND

Entry of non-permeable molecules into cells has been achieved using a variety of techniques, including use of lipofection, viral vectors, pore-forming proteins (e.g., alpha-hemolysin), as well as direct injection into the cytoplasm of cells. While lipofection and viral vectors are well suited to obtaining entry of nucleotides into cells, these methods are not amenable to the uptake of peptides, proteins and non-permeant molecules other than nucleotides. Pore-forming agents such as alpha-hemolysin are expensive and allow only small (<1000 d) molecules entry to the cell. Direct injection of molecules is not compatible with current high throughput practices. Furthermore, these approaches are not suited for use on primary cells in culture.

Entry of RNA, which generally are non-permeable molecules, into cells is used in the technique known as RNA interference (“RNAi”). Initially identified 1998, RNAi has rapidly become a very powerful tool for the discovery and validation of therapeutic targets in disease cell models. See, e.g., Fire et al., “Potent and specific genetic interference by a double stranded RNA in Caenorhabditis elegans,” Nature 391; 806-11 (1998). RNAi can be induced using chemically synthesized moieties of 22 nucleotides in length, or short interfering RNA (“siRNA”). See, e.g., Huppi et al., “Defining and assaying RNAi in mammalian cells,” Molecular Cell 17; 1-10 (2005). To produce gene silencing, these moieties require access to the cell cytoplasm where they complex with the RNA Induced Silencing Complex (“RISC”). Once bound to RISC, an siRNA that recognizes a cognate mRNA sequence is able to cleave that sequence into smaller fragments and therefore reduces the amount of mRNA for the protein available within the cell. Loss of function (“LOF”) of the protein encoded by the silenced gene can be used to determine the role of this protein in various aspects of cell signaling, cell growth and survival.

In order to improve the speed and utility of such techniques it is desirable to adapt the approach to employ high throughput screening (“HTS”) methods and systems to thereby allow rapid transfection and measurement of phenotypic outcome on cell culture models of disease. Several large libraries of siRNAs are commercially available, including libraries of siRNAs against every gene in the human genome. These libraries are providing tools for rapid identification of the role played by individual genes in the etiology of a disease. By knocking down a gene using RNAi and examining the outcome on a cell response, insight into the function of a gene can be obtained. A determination of the relevance of that gene as a therapeutic target can lead to new drug discoveries.

Using these large libraries of siRNAs, screeners will be able to examine the context under which a gene is important for the disease. For example, co-exposure with a known therapeutic agent during siRNA screening can reveal new sensitizing genes. Alternatively, upon identification of a point of vulnerability in a cell model using siRNA libraries in high throughput systems, it may be necessary to examine large numbers of small molecule compounds for their therapeutic potential within this same cell model, including where a gene of interest is knocked down by the identified siRNA in the presence of differing compounds.

Accordingly, a continuing and unmet need exists for new and improved platforms, including equipment, instrumentation and consumables, for performing such gene/siRNA/protein peptide delivery screens in high throughput, especially an automation-compatible transfection methodology that can be used to effect entry of oligonucleotides, proteins, peptides and other non-permeable molecules (e.g., some small organic compounds) into cells.

### SUMMARY

Described herein are platforms and consumables for performing gene/siRNA/protein peptide delivery screens in high throughput, including an automation-compatible transfection methodology that can be used to gain entry of oligonucleotides, proteins, peptides and other non-permeable molecules (e.g., some small organic compounds) into cells. Electrical stimulation is provided to cells, e.g., neurons or myocytes, in culture through application of pulsed fields directly to the cells in culture. The plate design is used in conjunction with a custom-designed circuit that allows the user to select pulse type, duration, etc. The methods described herein can be applied to transfection or stimulation of a variety of cells, including eukaryotic cells, such as mammalian cells (e.g., human, mouse, monkey, rat, etc.), as well as bacterial, insect or plant cells.

Heretofore known devices require manually clipping the upper electrode to the lower slide assembly and are not amenable to automation such as movement via a robotic arm or interfacing with automated liquid dispensers designed for microtiter plates. Furthermore, existing devices typically only have a single well and therefore in order to replicate observations, multiple individual devices need to be used, which is very time consuming. Additionally, minor variations in resistivity on the slide surface produce undesirable differences in the electrical response.

As further described herein, in a typical application an electrode is maintained at a fixed distance from the lower conductive surface, which uses a single confluent layer of ITO. Each well is separated by the use of the applied electrically conductive grid forming the ground plan conductor on the ITO sheet. Switching the upper electrode connections for the unused electrodes ensures minimal cross talk of the applied signal between separate wells. These unused electrodes can be switched so they receive no signal, are switched to ground (same as lower electrode), or switched to be in phase or out of phase with the applied signal.

The upper electrode and lid may be configured such that there is not an airtight seal between the lid (containing the upper electrodes) and the edges of the wells within the plate. This allows constant gaseous exchange with the surrounding air. When the plates are in an incubator this ensures continual growth and survival of the cells for many days in a 5% CO₂ environment that helps maintain the appropriate pH of the medium bathing the cells. The ability to remove the electrodes from the plate completely, and replace it with a standard microtiter plate lid, further improves the ability to grow cells before, during or after exposure to pulses from the stimulating electronics. Additional features include repetitive application of pulses with given pulse frequency (DC to 1 MHz), amplitude (0 mV to 10 V p-p or 200 VDC), varying time between applied pulse trains (0 seconds to many minutes).

By reducing the diameter of the upper electrodes and allowing movement of these in X, Y, and Z directions it is possible to selectively stimulate cells within each well of a plate. This can, in turn, be automated on HTS instruments or manual microscopes or even end-point readers by imaging the well using image analysis software to predict locations of cells, identifying the location of cells of interest within a well, driving an upper electrode to this location and lowering to the correct height over the cells, applying a stimulus to the cells beneath the electrode.

### DESCRIPTION OF THE DRAWINGS

FIG. 1 schematically compares electroporation of a suspension of cells with electroporation of cells adherent on a surface.

FIG. 2 is an exploded view of a microtiter plate assembly according to an example embodiment hereof.

FIG. 3 illustrates a microscope slide-based system according to an example embodiment hereof.

FIG. 4 illustrates a block diagram of example pulse stimulating circuitry.

FIG. 5A illustrates an example upper electrode assembly, and FIG. 5B is an example circuit diagram for electroporation using capacitor discharge

FIG. 6 illustrates an example software interface for controlling the application of pulses to a microtiter plate system.

FIG. 7 illustrates a setup for measurement of calcium flex from cells stimulated with electrical pulses.

FIG. 8 illustrates example pulse shapes that can be applied to upper electrodes.

FIG. 9 illustrates a side view of an example conductive plate.

FIG. 10 illustrates a side view of an example electrode assembly in use.

FIG. 11 illustrates an example circuit for controlling application of a signal to the upper electrodes.

FIG. 12 illustrates use of DC pulses to obtain electroporation and sample uptake into cells using siRNA silencing of GFP expression.

FIG. 13 illustrates movement of an example upper electrode assembly in the Z direction.

FIG. 14 illustrates an expanded view of electrode tips in accordance with an example embodiment hereof.

FIG. 15 illustrates a narrow upper electrode in accordance with an example embodiment hereof.

FIG. 16 illustrates an example open array transfection apparatus.

FIG. 17 illustrates an example block diagram for a DC voltage supply.

### DETAILED DESCRIPTION

In situ electroporation of cells, first described in the scientific literature several years ago, has shown utility in gaining entry of a variety of different molecular species into the cytoplasm of cells. See, e.g., Raptis et al., “Electroporation of adherent cells in situ,” DNA and Cell Biology 9, 615-21 (1990). This technique can be used to gain access of a variety of reagents into cells without significant loss of cell viability. Materials demonstrated to be taken up using this method have included non-permeant small molecules, double stranded oligonucleotides and small nucleotide derivatives, as well as peptides and proteins. According to such “in situ electroporation” methodologies, cells are cultured directly on an electrically conductive material that has been deposited on a glass substrate. An upper electrode is placed into a liquid bathing the cells and, by application of a brief electrical pulse, pores are generated within the cell membranes. By inclusion of a material (e.g., siRNA, miRNA mimic or inhibitor, PNA or DNA plasmid) in the bathing medium, electroporation permits such materials to pass through the pores and enter the cytoplasm of the cells. The pores generated are transient, and they rapidly (within two minutes) reseal, capturing the material of interest within the cell while maintaining cell viability.

According to a heretofore known method of in situ electroporation, cells may be grown on slides, with electroporation achieved using a customized apparatus that is cumbersome and difficult to manually operate. Recently, companies such as Applied Biosystems/Ambion (Austin, Tex.) and Harvard Bioscience, Inc. (Holliston, Mass.) have introduced electroporation equipment that is able to use microtiter plate-based formats. However, these approaches suffer from several drawbacks.

Such art-recognized electroporation systems are not automation friendly and typically require significant manual intervention. The electroporation systems have vertical electrodes within each well and therefore electroporation of cells must occur in suspension, which requires the cells to be transfected immediately after removal from the flask or other culture vessel. Such a requirement limits the timing of the transfection, and it may cause cells to be compromised by the removal from an adherent mode of growth thereby potentially demonstrating differential responses to gene silencing. Moreover, the electrodes are far apart within each well, which requires significantly higher voltages to obtain permeabilization, and such high voltages can kill a large proportion of the cells. Cells in suspension are evenly distributed across each well, and therefore some cells may be closer to the electrodes than to the middle of the well. A voltage applied for optimal transfection of cells at the center of the well may kill those closer to the electrodes exposed to the higher voltage, and the proportion of transfected cells to viable cells may be significantly reduced. Furthermore, the plates used in current designs have only 96 wells, and the design of the electrode assembly is not readily scalable to allow higher well densities (e.g., 384 well or 1536 well layouts). Additionally, the use of 96-well plates requires larger well volume and more reagent per well, which proportionally increases costs.

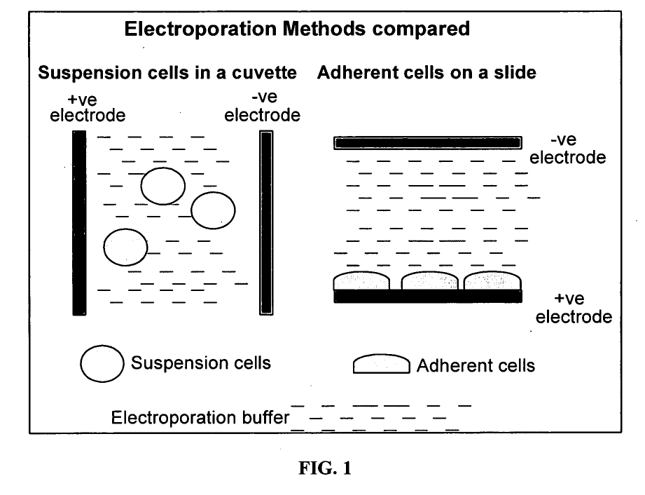

Referring to the attached Drawings, FIG. 1 illustrates a conventional in situ electroporation method on the left, and an example improved in situ electroporation process on the right. According to the in situ electroporation process shown on the right, the cells are maintained as adherent cultures in a monolayer on a lower electrode surface, whereas heretofore art-recognized methods as depicted on the left of FIG. 1, include vertical electrodes within a reaction vessel (e.g., a cuvette or well).

The adherent cell in situ electroporation process as illustrated in FIG. 1 and as further described here includes many advantages. For example, because cells grow as a monolayer on the lower electrode and the upper electrode can be introduced to just a few hundred microns above this surface, applied voltages can be significantly reduced because of the shorter distance between electrodes. Furthermore, all cells in the monolayer are exposed to an equal voltage thereby reducing variability across the cell population.

In an embodiment, the plates are compatible with existing HTS screening plate-handling equipment, as well as existing liquid-handling equipment. Similarly, the electro-permeabilization workstation may be designed and constructed to be compatible with HTS activities, such as automated plate changing with a robotic arm, which may be executed with software that allows the apparatus to interface with arms from several different manufacturers.

In order to be compatible with industry-standard instruments as used in high throughput screening, the adherent cell in situ electroporation apparatus is configured to selectively address wells within plates according to the standard introduced by The Society for Biomolecular Screening (“SBS”). Standard operations include adding reagent(s), washing and reading or collecting data. While microtiter plates may have various well densities (typically 6, 24, 48, 96, 384 and 1536-well plates), they all share the same footprint, as defined in American National Standards Institute (“ANSI”) SBS 1-2004 specification, which is incorporated herein by reference. Advantages of higher density plates include minimization of reagent volumes, reduced numbers of plates, and increased data density.

During electroporation, some parameters that impact pore formation and that are controllable during stimulation include: (1) the distance between upper and lower electrodes; (2) the applied voltage; (3) the duration of applied voltage; (4) the concentration of material to be introduced in the surrounding medium; (5) the number of cells within the electric field; and (6) the composition of the buffer. Where the external material to be taken up by the cells is an oligonucleotide (e.g., an siRNA, miRNA, a peptide nucleic acid (“PNA”) or a DNA oligonucleotide) the process is referred to as “electrofection,” i.e., transfection using electrically induced pore formation in cells.

### Microtiter Plates

Many analytical techniques use cells in culture to examine how these cells respond under certain physiological conditions. Such studies often seek to closely mimic endogenous physiological processes or stimulation to understand the mechanisms that are involved in disease or cell physiology within the intact organism. Over the years, the ability to culture cells has been enabled by the introduction of sterile plastic flasks and other culture ware, as well as specialized growth media in which cells can divide and propagate. Many experiments are still performed in Petri dishes, which are large plastic dishes on which cells typically adhere to the bottom surface and can be imaged using microscopy or biochemical measurements.

For high throughput experimentation, a standard multi-welled plate or microtiter plate has been adapted. These plates have a standard size defined by the Society for Biomolecular Screening. They are typically made of polystyrene and the base of the plate may be polystyrene, polycarbonate or even glass and may be provided with or without a plastic lid that sits on top of the plate to protect the cells from external contamination by ambient bacteria, fungi, etc. A plate may contain 96, 384 or even as many as 1536 wells within this standard footprint, although other configurations are available. By way of example, a 96-well plate is laid out as an 8×12 matrix with wells spaced 9 mm on centre. A 384-well plate requires four times as many wells within the same footprint, so these wells are in a 16×24 matrix and are 4.5 mm on centre.

An advantage of a standard plate layout and geometry includes ease of design and integration of automated instrumentation that can handle plates and perform distinct operations on them despite these plates coming from multiple manufacturers or being made from various plastics or other materials. Liquid dispensers, pipetters and readers are designed to be able to add liquids or read from wells of these plates independent of the manufacturer. Another advantage of automation and standard size of these consumables is that many robotic arms are available that are designed to transport these plates from one instrument to the next where a defined series of liquid additions, washing and reading can be performed in a sequence leading to the ability to perform screening in high throughput. Robotic platforms of this nature work around the clock and perform each step on a rigorous time schedule ensuring improved reproducibility of the data.

### Construction of Electroporation Plates and Electrodes

The electroporation plates are manufactured to comply with the SBS microtiter plate standard and therefore have a defined geometry for well layout, skirt height, and similar characteristics. This design ensures compatibility of the plates with existing high throughput screening automation and allows the electroporation plates to be manipulated by industry standard robotic automation during the screening process.

In an example embodiment, electroporation plates have a lower surface usually made of glass and optically clear to allow visualization of the cells within each well from below the plate, coupled to an upper gasket assembly designed to mimic the layout of a standard multi-welled (e.g., 384-well) microtiter plate. The glass (or a functionally equivalent material) has an electrically conductive layer of indium tin oxide (“ITO”) vapor deposited on the upper surface that is capable of contacting the cells.

A gasket material is applied at the locations of each of the well walls. This gasket material may be made of copper foil, or it may be a conductive ink applied via silk screening or similar method onto the ITO coated surface. Further examples include vapor-deposited metal surfaces such as chromium, gold, silver, or even conductive epoxy materials applied through deposition using a syringe and needle dispenser assembly.

An electrode assembly is configured to provide electrical conductivity between the ITO layer on the glass and the upper electrodes. Distance between the upper electrodes and lower conductive surface can be altered between about 150 μm to a maximum equivalent to the height of the well. During operation, a minimal distance between the upper electrodes and the lower conductive surface minimizes the voltage required for electroporation, and this reduced voltage may advantageously provide for a greater number of viable cells after stimulation.

Additionally, 96-well or 384-well plates having an upper plastic assembly glued to a lower plate of glass that then forms the bottom of each of the wells are commercially available, including Corning Costar® (COSTAR is a registered trademark of Corning, Inc.), Greiner Bio-One and MatriCal, Inc. multi-well plates.

The ITO-coated substrate may be obtained from Delta-Tech, Chomerics, or from another vendor. The coated glass is typically cut to the appropriate size to fit the bottom of the microtiter plate, epoxy or conductive ink is deposited on the glass using either an X,Y motorized applicator or through the use of screen printing. The substrate is then attached to the bottom of the plastic extrusion that forms the upper portion of the plate using, e.g., either a silicon adhesive or a suitable UV-curable adhesive. Plates are sterilized after manufacture by, e.g., exposure to gamma radiation. Sterilization allows extended times for cell culture on the plates and minimizes the risk of contamination by bacteria and the like during the course of electrofection.

In an example embodiment, plate manufacture includes attaching the glass (precoated with the ITO electrically conductive surface) to a pre-molded plastic plate. A plate is typically turned upside-down to receive the glass that is glued in place using UV-curable adhesives. The electrically conductive surface is located so that it is facing upwards in the completed plate.

Upon attachment of the upper plastic assembly to the conductive substrate, an electrical connection is made between the conductive gasket and a metallic strip at the edges of the microtiter plate. In an embodiment, a metallic strip is formed around the bottom edge of the plate skirt such that, when the plate sits flat on a metallic surface, there is a continuity in electrical conductivity between the lower electrode (ground plane) and the external metallic surface. This then forms the lower electrode assembly.

### Well-Less, Open Array Systems

The electroporation systems described herein also include well-less, open array systems, which may be used to increase the density of successful transfections. As discussed elsewhere herein, a microtiter plate-based system has many advantages, e.g., the capacity to examine multiple samples in parallel. In certain circumstances, however, microtiter plate-based systems may not be optimal. For example, with microtiter plate-based systems the number of samples that can be tested is limited by the number of wells available on a plate. To test larger numbers of samples, multiple plates must be used, and this typically requires access to automated equipment to allow efficient processing of the plates. Although they are uncommon, pipetting errors to a single well can result in data loss and necessitate repeating tests to minimize the effect of such a loss. Additionally, cells within each well of a plate may be exposed to differing micro-environments and result in “edge effects.” The amount of sample to be electroporated is higher based on the increased number of samples used and the volumes within the wells, and this in turn increases costs.

To address some of these issues, an electroporation platform including an open (well-less), array-based, format can be used. Arrays, e.g., DNA, RNA, or proteins/peptides, can be used with such a system. A well-less system does not contain individual wells within which the electroporation system localizes reagents. Instead, an array-based system relies on the deposition of small spots of material to be examined onto the lower conductive surface in such a way that, upon seeding cells on top of these spots, the material deposited is not immediately washed away and is not taken up into the cells until an electrical pulse is applied.

Advantages of such open-array, well-less systems include reduced use of the reagents (siRNA, DNA, protein, peptide, etc.). Also, small spots may be used, e.g., 300 μm diameter at 500 μm center to center spacing, which permits over 40,000 spots to be deposited within the same area (85 mm×120 mm) as a microtiter plate. Therefore, an entire genome may be examined in a single “plate.” Additionally, cell addition occurs across the entire array does not require a single pipette tip be used per “well;” cells are distributed evenly across the entire array surface and this minimizes the effect of localized micro-environments on cell growth and behavior.

Temporal control of material uptake by the cells is afforded by controlling when electric pulses are applied. In order to prevent the uptake of the siRNA (or other materials) into the cells prematurely, reagents are deposited onto a surface that is able to neutralize the charge of these moieties but that is sensitive to the change in applied voltage such that the application of a voltage between the electrodes reverses the charge-charge interaction between the reagents and neutralizes the charge on the chemical reagent. Release of the siRNA from the chemical and the positive voltage directs the deposited siRNA from the lower (-ve voltage) surface towards the positive electrode (upper). As the material moves through this electric gradient, it encounters the cells where it can gain entry through the pores formed from the initial electric pulse.

Consequently it is feasible to coordinate the entry of thousands of siRNAs as discrete spots on a surface into cells growing as adherent layers on this surface. Such chemical surfaces may include polypeptides (e.g., polylysine, where all amino acids are the D-enantiomer or all are L-enantiomer or a mixture of both). Alternatively, the polypeptide could be a copolymer of histidine, lysine, and arginine moieties. Similarly, the polymer could include histidine and lysine residues synthesized as described in Lenq et al., J. Gene Med. 7(7), 977-86 (July 2005), which is incorporated herein by reference.

Furthermore, the surface adherent material may be only weakly charged when siRNA is complexed with it and, upon application of a voltage, the charge is altered to allow repulsion of the siRNA (which is negatively charged) from the complex. Such a vehicle may include poly (D,L-lactide-co-glycolide) copolymer (“PLGA”) nanospheres. Using methods described by Ma et al. and Wilson et al. it is envisaged that siRNAs can be spotted onto a DODAP polymer matrix applied to the lower electrode surface. This matrix has the advantage that when siRNAs (or other negatively charged species) are bound to it, the overall charge is neutralized. Application of a voltage to the mixture then releases the siRNA from the matrix.

Microarrays of siRNAs (or other reagents to be transferred into the cells) can be electroporated in situ into the cells growing on top of the siRNA spots. Previous publications (Mousses et al.) have shown that this approach is feasible when used with a lipofection-based approach by combining the siRNA material to be spotted with the lipid along with gelatin and fibronectin to minimize loss of the material. Arrays of material were printed using a microarray spotter and localized transfections were observed. Other publications (see, e.g., Erfle et al.; and Conrad et al.), have also demonstrated the use of lipid-based transfection to afford uptake of materials into cells on such arrays.

However, while this approach apparently works well for siRNAs, it may be less useful for DNA, plasmids, etc., and it is unlikely that lipid-based arrays will have broader use for gaining access of peptides, proteins, antibodies and other non-permeable molecules into cells. The lipid, for example, may not be able to mediate access of such large molecules into cells and may even denature the material to be transfected.

The use of gelatin, agarose derivatives and other suitable materials may be used in a cocktail that, when admixed with the spotted material, prevents solubilization into the general media when cells are deposited on top of the spots. However, upon application of an electrical pulse to the samples, the samples must be released from the substrate, and, thus, allow entry of materials into the cells immediately in the vicinity of the spots through pores formed in the cell membranes upon exposure to the pulse.

### Biological Effects of Electric Stimulation

The systems described herein may be used to bring about a variety of biological effects, including, among others, (1) physiological stimulation, including mimicking endogenous electrical stimuli of cells in vivo; (2) low electric field stimulation (“LEF”), which permits uptake of external molecules into cells or induce responses in traditionally non-electrically stimulated cells; and (3) electroporation of cells (obtained using high voltage DC pulses or using applied frequency pulses to the cells), which facilitates entrance of external molecules normally prevented from entry by the plasma membrane.

### Mimicking Endogenous Electrical Stimulation in Cells in Culture

For many cell types used in biological experiments a suitable medium is all that they require to mimic the endogenous milieu that they would experience within the source organism (e.g., hepatocytes from the liver). However, many other cell types are not only exposed to hormonal or chemical stimuli but are also exposed to varying electrical stimuli. Such cells include neuronal cells or muscle cells. Neuronal cells can either be derived from primary culture or be immortalized so that, given the appropriate medium, supplements (e.g., serum), temperature and humidity, they will continue to divide for multiple generations without issue (e.g., rat PC12 cells).

These cells can respond to externally applied neurotransmitter molecules (GABA, glutamate, serotonin, etc.), which regulate their sensitivity to further transmitter release. They can also respond to small pulses of electricity as would normally be experienced during nerve depolarization within the organism. Similarly, muscle cells (or myocytes) are able to respond to electrical stimuli, which induce the shortening of the muscle fibers leading to muscle contractions. Thus, for either of these two cell types (and even for non-electrically stimulated cells types, as described elsewhere herein) the ability to provide electrical stimulation in multi-welled microtiter plates provides a closer mimic to the endogenous environment these cells are exposed to in their native environment.

Because such electrical stimulation has marked effects on endocytosis and exocytosis of neurotransmitters within neuronal cells, this may have significant effects on the ability of cells to take up exogenously applied reagents (e.g., small molecules with potential drug-like properties) than in un-stimulated cells. As a result, assaying the effects of a drug candidate on un-stimulated cells may lead to misleading conclusions for the candidates' actual efficacy and potency. The ability to mimic endogenous electrical stimulatory mechanisms during the culture steps and high throughput screening phases can more accurately predict behavior of a drug candidate in vivo than when compared with cells that were not stimulated during exposure to the drug candidate.

Accordingly, the systems and methods described herein may be employed to provide such electrical stimulation during experimental steps and more closely mimic endogenous electrical stimulation of cells in vivo. For example, exposure of either neuronal or muscle cells to sinusoidal or square wave pulses with a frequency of about 0.1 Hz to several KHz and amplitudes in the milli-volt range mimic endogenous stimulation (or tetanic stimulation) in vivo. Furthermore, multiplexed stimulation of such cells using either sine wave, square wave or even saw tooth patterns with frequencies between about 0.1 Hz and 1 MHz with amplitudes between about 0 mV and 10 V (peak to peak) may be achieved. Varying the height of the electrodes above the lower conductive surface allows further alteration of the exposure that the cells observe, e.g., variation in apparent V/cm.

Physiologic stimulation with applied electronic fields has been demonstrated for a variety of tissue types. For example, Berger et al. showed that continual electrical stimulation of adult ventricular myocytes for several days with frequencies between 0.1 Hz and 7 Hz maintained the cells in a state more closely mimicking freshly isolated cells. Kimura et al. demonstrated the ability to produce neurite outgrowth from PC12 cells growing on ITO electrodes when stimulated with rectangular pulses with amplitudes of 200 mV and frequencies between 50 Hz and 1 KHz. Optimal neurite outgrowth was observed at 200 mV and 100 Hz stimulation. Blockade of this effect by LaCl₃ suggested that the morphological response was induced by the potential shift in the proximity of the cellular membrane resulting in alterations in calcium uptake. Similarly, Holt et al. showed that a 1 Hz signal applied for 24 h to myocytes in culture enhanced their mechanical properties as well as calcium transients, and electrical stimulation may be important when studying the function of adult ventricular myocytes. Additionally, alterations in electrical potentials experienced by tissue are relevant to the vasculature as it relates to blood flow, as well as wound healing and tumor development and growth (Zhao et al., 1994). Accordingly, electronic stimulation of cells derived from any of the foregoing tissue types may be beneficially subjected to the methods and systems described herein.

### Low Electric Field Stimulation of Cells in Culture

The size and duration of the pulses applied across cells can be altered in such a way as to provide low electric field (“LEF”) stimulation of the cells. One embodiment of this approach can be used in a multi-welled device to induce uptake of externally applied reagents such as siRNA, shRNA, DNA, proteins, peptides or other molecules, e.g., calcium dyes, organic macromolecules or small molecule drugs. For example, LEF can be applied to either neuronal or muscle cells, and it has also been shown to be useful in gaining access of a number of materials across the cellular plasma membrane.

Electrical stimulation has been shown to induce angiogenesis (new blood supply formation) in vivo and application of electrical pulses of small physiological magnitude to endothelial cells in culture directly stimulated VEGF production form these cells (Zhao et al, 1994). The amplitude of the applied voltage was 75-100 mV/mm and this was shown to induce a physiological change in the cells including reorientation, elongation and migration of the endothelial cells in culture. Thus, the systems and methods described herein may be advantageously used in high throughput experiments aimed at using applied LEFs to initiate and guide angiogenesis.

Experiments examining the effects of gene silencing (or overexpression) or the effects of exogenous reagents (small molecules, antibodies, etc.) can be performed in LEF-stimulated cells in culture in a high throughput manner. For example, Qiu et al. showed that stromal cells from rat bone marrow could be induced to change morphology by altering the charge on the electrode surface on which they were cultured. Applied voltages (in this case, DC) were between 0.7 and 1 V. Positively charged ITO as a substrate was able to promote cell adhesion while decreasing cell spreading. Accordingly, using the systems and methods hereof, parallel evaluation of similar applied electric fields on cells in culture is possible. Furthermore, multiple replicates of the same conditions may be evaluated in a single experiment, thereby raising the quality of the statistical results obtained.

In studies on cancer cells, it has been shown that exposure to low electric fields can enhance the effect of drugs such as doxorubicin against multidrug resistant cell lines. Janigro et al. showed that an AC frequency of 50 Hz for three days enhanced sensitivity of a variety of cancer cells to the drug due to alteration in expression of cellular drug resistance mechanisms. Results were obtained for C6 (glioma cells), PC3 (prostate cancer cells), H1299 (lung tumor cells), H1080 (fibrosarcoma), and SKOV3 (ovarian cancer) cells.

Low electric field stimulation has also been shown to alter transcription factor expression patterns in muscle cells (Ircher et al.) and to increase the activation of cytochrome c genes in neonatal cardiac cells (Xia et al.). Additionally, DC electric fields have been shown to induce calcium response and growth stimulation of tumor spheroids (Sauer et al.). Similar work also showed how application of a single electric pulse to such spheroid tumors could induce an increase in intracellular calcium due to release of calcium from intracellular stores (Wartenburg et al.). Consequently, because the systems and methods described herein use, for example, a plurality of transparent lower electrode surfaces, they can be coupled with suitable readers such that pulses are applied while imaging to observe such alterations directly in real time. Accordingly, the systems and methods described herein may be advantageously employed in the study of the foregoing tissue types.

In a similar manner, longer term experiments can be performed where repeated measurements of physiological changes in the cells within each well can be made by taking measurements using either standard plate readers (measuring fluorescence, luminescence, etc.), or at a single cell level by combining this with subcellular imaging using a microscope or automated high content screening platform. Furthermore, in primary hippocampal neuronal cells exposed to low electric field stimuli, Balkowiec et al. were able to observe BDNF release from these cells and could not mimic this effect by depolarizing the neurons using high K⁺ levels (50 mM). This stimulation resulted in calcium entry through N-type calcium channels. The amount of BDNF release correlated with the frequency used and increased between 5 Hz and 100 Hz. The direct application of signals of varying frequencies and amplitude to the cells resulted in depolarization induced release of transmitter, whereas altering K⁺ had no effect. Thus, the systems described herein provide improved methods for evaluating neuronal cells in culture (in vitro) and may more closely mimic the physiological state of the cells in vivo.

The ability to provide electrical pulses to cells in culture while having the ability to image the cells from below the plate can also have benefits in examining the role that such stimulation has on morphology changes induced in these cells. For example, Fields et al. showed that patterned electrical activity could increase neurite outgrowth from primary mouse sensory neurons. Schmidt et al. also showed the ability to stimulate neurite outgrowth in rat PC12 cells upon electrical stimulation. Reiher et al. had shown that an interdigitated electrode assembly could also be used to induce stimulation of neuronal cells in culture on these lower electrodes.

Accordingly, the systems and methods hereof have utility in parallel processing and identification of optimal conditions required for tissue engineering. For example, application of LEFs to cardiac myocytes results in cell alignment and coupling as well as increased contractile strength in cultures while improving ultrastructural organization of the cultured cells (Radisic et al.). External electric fields were also shown to induce morphological changes in human skin cells cultured in vitro (Dube et al.). Thus the systems of methods hereof are useful in studying non-electrically excitable cell types, as well as the electrically excitable neuronal and muscle cells.

### Electroporation of Cells in Culture

Electroporation is a means for gaining entry of foreign (large) molecules from the extracellular region to the cellular cytoplasm (Weaver, J C 1995, Ramos et al., 2000). Electroporation causes permeabilization of a plasma membrane by a pulse of applied voltage. Consequently, electrophoretic transfer of DNA through these membrane pores occurs (Ramos et al., 2000). Higher amplitude pulses applied as discrete single pulses of fixed duration rather than as a continuous sine/square wave pulse train are used to induce electroporation. Under such circumstances, the threshold of the amplitude of such pulses can overcome the electrical resistivity of the cellular plasma membrane and allow induction of small pores within the membrane. These pores allow uptake of molecules in the external medium into the cellular cytoplasm. Such molecules may include siRNA, shRNA, DNA, plasmids, proteins, antibodies, peptides or other molecules, e.g., calcium dyes, organic macromolecules or small molecule drugs.

This process, known as “electroporation” relies on the fact that, with pulses of a given amplitude and short duration, the pores, which allow entry of foreign materials, can reseal without significantly harming the cell. Continued culturing of cells exposed to such electroporation can be achieved using the devices described herein. Entry of an exogenous reagent into the cytosol of the cell can be evaluated for its effect on physiological parameters or phenotypic alterations in the cellular behavior. Using the multiwelled approach where each well is treated identically or under different stimuli allows multiple parameters to be evaluated in parallel on cells grown under essentially identical conditions.

Raptis et al. (1995, 1998) demonstrated the ability to electroporate cells growing on a conductive ITO surface (Raptis et al, 1995, Raptis et al., 1998). The device used a single large stainless electrode to be able to provide a pulse to a single chamber. The design of the chamber was such that cells could not be maintained within it for long durations since they could not adequately exchange gases with the external environment and, due to the geometry of the gasket assembly, the amount of medium bathing the cells was very low and would be rapidly exhausted. The latter authors used a large DC pulse (50 V×4 pulses to an area 32×10 mm) to electroporate the cells.

Another method of electroporation was shown using gold electrodes on which cells were attached and the application of an AC pulse (Wegener et al., 2002). Pulsing had to be stopped during readout in this system since the same electrodes were used for both stimulation and measurement of electrical impedance changes induced in the cells. AC voltages of 40 KHz with 3 V amplitude and 100 microseconds in duration could result in uptake of the non-permeable dye Lucifer yellow into kidney cells growing on the gold electrodes. Low voltage pulsing electric fields have also shown to be able to enhance uptake of materials into cells (Antov, Y. et al, 2005).

Plasmids have also been effectively introduced into cells in culture by inserting two aluminum electrodes inserted directly into a Petri dish where the cells are cultured (Takata, K et al., 2003). GFP plasmids were used to monitor expression of green fluorescent protein upon transfection. Using this apparatus, transfection in 3T3-L1 fibroblast cells, HeLa cells, Cos-7 cells and MDCK cells were achieved. The electrodes were spaced such that high voltages were required for electroporation (250 V with duration of 100 milliseconds and 10 pulses applied).

As described herein, limiting the distance of the upper electrode over the lower electrode surface permits electroporation across the cell field with lower voltages (under 60 V). The transfer of material might be expected to be low because the field applied to the cells was parallel to the surface of the cells on the coverslip. Efficiencies may be expected to vary based on cell distance from the electrodes; however, it has now been found that the distance from the upper electrodes to the lower electrode surface on which cells are growing is constant across the whole field. Consequently, yields of transfection efficiency are expected to be better across all fields within a well. The ability to parallelize the process using a multiplexed electrode assembly where the distance from the upper electrodes to the lower electrodes is constant provides a better control than the described systems where individual chambers were set up. Furthermore, Takata et al. point out that adherent cells may not behave appropriately under electroporation in cuvettes where electrodes are vertical but may behave better if electroporated while adhering to a surface (Takata et al., 2003, Yang et al., 1995).

### Substrates

Either glass or other transparent substrates (polycarbonate, polystyrene, quartz, borosilicate) may be used as substrates upon which a coating of ITO is made. Alternatively, the conductive surface itself may be non-transparent and deposited on other conductive materials, e.g., the surface may be ITO, copper, gold, silver or alloys thereof, and these could be deposited on other metallic and conductive surfaces such as gold silver copper or nickel. Alternatively the conductive layer could be deposited on non-conductive or semi-conductive materials such as plastics (polystyrene, polycarbonate, glass) or semi-conductive materials such as silicon dioxide, gallium arsenide or mixtures thereof.

At very low resistivity it is possible to obtain a layer of ITO on these substrates that is itself transparent and yet provides a conductive layer. Consequently, a preferred embodiment includes a thin, transparent layer of ITO on glass or polycarbonate, which allows the user to be able to observe the location of cells within a field within each well.

### Upper Electrodes

The upper electrodes can be of a dimension similar to that of each well in which they are inserted or, alternatively, they can be of a very much smaller dimension. They typically are held in space at distances corresponding to the spacing between wells in the plate by a non-conductive material. The electrodes themselves can be made of any conductive material, although they preferably are inert and are able to conduct DC and frequency pulses with minimal deleterious effect on the electrode.

In an example embodiment, these electrodes are stainless steel (or another conductive metal) and may be plated with gold. The electrodes are contacted with an electrical supply via electrical traces applied to the non-conductive gasket material. Such electrical traces can be etched into the gasket from copper clad phenolic material using methods known in the art. The copper traces are connected to the electrical supply via an external electrical connector that can be attached to the board by soldering to points of contact and holes in the gasket. Thus, in an embodiment, the voltage applied to the cells (located directly on the electrode surface) can be varied either by directly reducing the applied voltage or by varying the height of the electrode within the well (moving the electrode further from the lower electrode surface will have a similar effect to reducing the applied voltage at the original height and vice versa).

If the electrodes are of a much reduced diameter relative to the size of the well then stimulation can be localized to specific regions of the well by moving the upper electrode within the well. This can be performed manually by moving the upper electrode in an X, Y or Z direction or, alternatively, using an automated stage driven by a set of motors to produce the same effect. This approach may be used as follows: Cells growing on the lower electrode are imaged under a microscope (or similar imaging system, e.g., via a video/camera capture device or using a high content screening instrument) and regions of the well containing the cells of interest are located. The electrode can then be moved to be directly over the cells of interest and the pulses applied at this location.

In a single culture system (e.g., neurons in culture) this method can be used to stimulate a set of neurons directly and then visualize the impact of this stimulation on other neurons that are directly in contact with the stimulated neurons but are outside of the range of the electrical pulses. Another example, in a co-culture model, uses a mixture of neurons and muscle cells within the same well. Under the imaging system, the location of a neuron impacting a muscle cell could be identified and the upper electrode placed directly over top of the neurons of interest. The effect on the muscle cells can then be evaluated during or after stimulation of the neurons.

Because the device uses a multi-welled enclosure for the cells, each well can be examined independently. Thus additional parameters can be evaluated outside of the effect of stimulation. An example of this relates to the study of the effect of a drug or protein on a phenotype within the wells both in the absence of stimulation and in the presence of a stimulus. The plates can be placed back into a tissue culture incubator after stimulation or can even be stimulated whilst in an incubator. This facilitates appropriate growth conditions for the cells subsequent to the stimulus and allows the longer term effects of these treatments to be examined. The open well design allows gas exchange with the media in which the cells are growing in each well.

### Stimulation of Cells in Situ

Stimulation of cells on the electrode surface is enabled by using a microcontroller to regulate the application of electrical pulses from the pulse circuitry (sine wave generator or DC voltage source) to the wells of interest. The direction of the pulses to specific wells may be achieved by means of a set of relays and digital switches controlled by the microcontroller. These switches are on-off-on switches such that the direction of the switch enables electrical connection of the lower electrode surface within each well either to electrical ground (in the case of DC pulses) or to the negative signal (when using oscillating signals where the lower electrode must allow negative voltages to be applied.

During the application of DC pulses to each set of wells it is important to ensure that neighboring wells do not see an induced potential. In order to minimize this, all electrodes that are not actively stimulating the cells are switched to ground while the stimulating electrodes can be held at the required potential. The time of each set of pulses and the delay between pulses may be controlled by the microcontroller. The microcontroller can activate the switches within nanoseconds and the minimum time interval for application of pulses is two microseconds. Maximum pulse time is over 60 seconds per pulse train but many applications require pulses in the range of milliseconds to seconds per pulse train. Delays between pulse trains can also be varied to allow the cells to recover from the stimulus before reapplication.

Electrical pulses may be applied uniformly to non-adherent cells. Because non-adherent cells do not attach to the electrical surface it is often difficult to image such cells since they remain diffusely arranged within the medium in each well. In standard electroporation devices these cells are electrically stimulated by placing a suspension of these cells between electrodes that are maintained in a vertical orientation. The cell suspension is triturated or otherwise manipulated to ensure a uniform distribution of cells within the medium.

However, when this cell suspension is placed between the electrodes some of the cells will inevitably be closer to each of the electrode pair while a smaller population is maintained at the centre of the gap between the electrodes. Consequently, the cells will experience varying levels of electrical stimulation depending on their distance from the electrodes. In many of these systems, the degree of uptake of samples by such cell exposed to electrical pulses in these systems only reaches 30-40% transfection efficiency.

In a beneficial embodiment that over comes this deficiency found in the prior art, the lower electrodes are horizontal and, consequently, cells that ordinarily grow in suspension will, under the force of gravity, eventually will be deposited on the lower electrode. This process can be accelerated by the use of a centrifuge where the plate is located in a swinging bucket such that centrifugal/centripetal forces are applied directly towards the lower electrode surface. Cells in suspension will be sedimented directly onto the lower electrode. After centrifugation, the plate is placed on a horizontal surface and the upper electrode assembly lowered into place such that there is a defined gap between the upper electrode surface and the lower electrode with a layer of cells present.

If the cell numbers are maintained at a low enough cell density within each well, a monolayer of the cells will be deposited on the lower electrode. The distance between the upper electrode and the lower conductive plane is constant between different wells and the cells are immediately adjacent to the lower electrode. All cells within a well will experience a similar electrical force based on the amplitude of the applied electric field and the distance between the upper electrodes and the lower conductive plane.

The nature of the applied electrical field can be, among other things, (1) a DC voltage, where the upper electrode is the positive electrode and the lower conductive plane is held at electrical ground; (2) a DC voltage, where the upper electrode is the negative electrode and the lower conductive plane is held at a positive potential relative to the upper electrode; (3) an oscillating voltage at varying frequencies, where the upper electrode is the positive electrode and the lower conductive plane is held at electrical ground; (4) an oscillating voltage at varying frequencies, where the upper electrode is the positive electrode and the lower conductive plane is allowed to go to negative potentials relative to electrical ground; (5) pulses of varying waveforms including a saw tooth shape, a square wave pulse where the lowest voltage applied is 0 V or where the lower voltage applied is the negative equivalent of the applied positive potential, and the waves could also be sine waves.

Additionally, the amplitude of the applied pulses can be varied by (a) varying the electrical parameters on the equipment used to generate the signals; (b) varying the height of the upper electrodes from the lower conductive plane; (c) varying the resistivity of the medium in which the cells are being stimulated; or (d) varying the amount of ITO on the lower conductive surface, which will vary the resistance of the electrode

Each of the electrical waveforms applied to the cells between the upper and lower electrodes can be altered in amplitude (0 V to 200 VDC or 0 V to 10 V p-p using frequency) or in frequency (between 0 Hz and frequencies limited only by the external pulse generator (typically 1 MHz)). In an example embodiment, pulse amplitude and frequency are chosen dependent on the end result required of the experiment. For example, for electrical stimulations of neuronal cells aimed at mimicking those pulses seen in cells in vivo, a frequency may be applied to the cells and the amplitude may be 100 mV and the frequency may be below 20 KHz. The duration of the applied electric field may vary between several milliseconds and many hours. For electrical pulses aimed at creation of transient pores in the membranes of cells the two methods that could be used include applied frequency pulses and applied DC voltage.

Upon generation of pores in the cells using either method, it is necessary to permit the reagents to be transfected to be driven towards the cells. If the lower electrode is the negative or ground terminal in the electrical system, then a positive potential on the upper electrode would be expected to attract negatively charged moieties such as RNA (e.g., siRNAs, shRNAs, miRNAs) or DNA (e.g., antisense molecules or plasmids). This results in a concentration gradient of the materials (e.g., siRNAs) and migration of these species by electrophoresis towards the cell layer.

In another embodiment, it is feasible to alter the DC potential on the lower electrode such that it is highly negatively charged relative to the upper electrode. This in turn will serve to further repel the negatively charged molecules such as siRNAs, RNA, miRNA, DNA, etc. away from the lower conductive plane and through the cell layer and towards the upper electrode.

While the application of a DC potential of low magnitude might drive the reagents towards the cell layer and help increase uptake into the cells (after pore formation), another embodiment includes applying an oscillating potential such that the charged moieties are driven from the lower conductive surface towards the cell layer and, by varying either the frequency of the applied AC voltage or by varying the amplitude of the signal (or both) that it is feasible to have the reagents move firstly towards the cell layer and then be driven back towards the lower conductive plane such that the net result is that the moieties spend a considerably longer time in the vicinity of the cell layer allowing increased localized concentrations in this area and therefore have higher uptake into the porated cells.

### Surface Treatment

In addition, surface modification to the surface (e.g., ITO) can allow selective binding of different biological species. DNA, RNA and siRNA, miRNA reagents are negatively charged species. They are typically transfected into cells using a cationic lipid. In the case of the ITO coated surfaces, it would be helpful to have a positively charged surface that could bind the negatively charged molecules. Upon application of a potential (+ve charge on upper electrode, negative voltage on the ITO surface) between the surface and the upper electrode the effect would be two fold. First, the negative voltage on the lower electrode would act to repel the negatively charged siRNAs and, second, the voltage difference would promote an electrophoretic force causing the migration of the siRNAs away from the lower surface and into the pores generated in the cells upon application of the high voltage.

Such a charged surface should ideally also be electrically conductive and transparent. Such a surface can be obtained with polypyrrole. Polypyrrole is positively charged and has “holes” where negative ions can be “doped” into the structure. siRNAs or miRNAs or other negatively charged species could be incorporated this way, and they are held by the charge interaction until a voltage was applied.

To provide localized regions for siRNA deposition and cell transfection, polyvinyl alcohol (“PVA”) could be deposited on the ITO surface and small holes in this surface induced by spotting sodium hypochlorite onto the immobilized PVA. This oxidizes the surface and exposes regions that are able to bind proteins and encourage cell growth.

### In Situ Cell Stimulators

Use of an apparatus as described herein, which may be referred to as an in situ cell stimulator (“ISCS”), can provide a constant level of electrical stimulation to cells maintained in culture. Such cells can include neuronal cells or muscle cells for example but evidence exists that many cell types can increase production of proteins when grown in the presence of an electric field.

The ICSC uses a process known as “field stimulation” to provide electrical stimulation to the cells during culture. The design allows multiple samples to be exposed to the same stimulus in parallel and also allows for control wells to be observed where cells were grown in the absence of the stimulus. One attribute of an ICSC system is the fact that the cells may be grown directly on the lower electrode and the design uses a well spacing of either 9 mm or 4.5 mm between adjacent wells, a format akin to 96-well or 384-well microtiter plates respectively.

Thus the cell trays can be placed into the same equipment used for high throughput assays to read out the effects on cell viability, neurite outgrowth, etc. using anywhere from a simple microscope to visualize the cells to a plate reader to monitor fluorescence or luminescence signals from the cells (e.g., representing expression levels of a GFP or luciferase tagged protein). Because the lower electrode is transparent (typically including a very fine layer of indium tin oxide vapor deposited onto glass or other transparent substrates (e.g., polycarbonate or other plastics)), the cells can be visualized during the process of stimulation. This allows measurement of depolarization induced phenomena in neuronal cultures, e.g., calcium flux.

Because visualization and stimulus production are non-invasive techniques, the apparatus allows for kinetic readouts where effects of a stimulus can be monitored by repeat measurement of the effect on the cells using a suitable readout. For example, effects on neurite outgrowth from neuronal cells may be monitored in response to a continued or intermittent stimulus of voltages at various frequencies, amplitude and duration. Since neurite outgrowth can take some time to develop, the cells could be evaluated using a suitable High Content Screening system with appropriate image analysis algorithms, e.g., the neurite outgrowth imaging bioapplication run on the Cellomics ArrayScan V (Cellomics, Pittsburgh, Pa.; www.cellomics.com).

The system includes a frame (for example, plastic or metal) that defines a series of chambers (wells) where the spacing between the wells is ideally consistent with the spacings used in either 96-well or 394-well plates. For 96-well plates the wells would be at 9 mm center to center and for 384 they would be 4.5 mm center to center. The number of wells may be anywhere from a single well to a multiplexed array. Ideally, the array could have 16 wells at 9 mm spacing where there are 8 rows and 2 columns and this frame would fit over top of a substrate that was the size of a microscope slide (1″×3″). In another example, a 96-well version may have 8 rows and 12 columns at 9 mm center to center spacing and a 384-well version may have 16 rows×24 columns at 4.5 mm spacing. A preferred format has dimensions to match the prescribed geometry of a standard microtiter plate as defined by the Society of Biomolecular Screening (www.sbsonline.org).

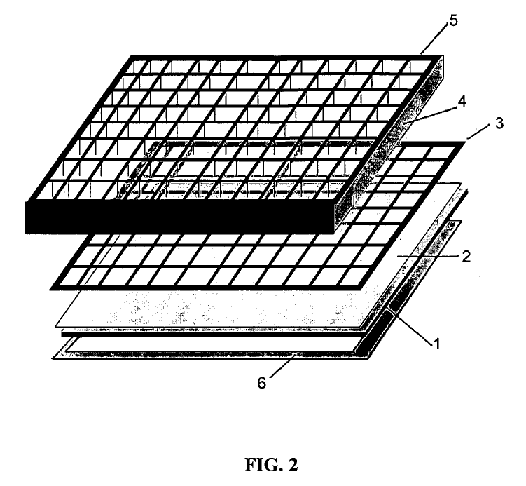

Referring to the attached Drawings, a substrate typically including a transparent media (for example, glass or plastic), typically less than 1 mm thick, on one side of which is deposited a thin layer of an electrically conductive substance that is, itself, electrically conductive and yet still transparent. The substrate is attached to the lower face of the frame in a manner that allows the electrically conductive surface to be directed towards the interior of each well and whereby liquids placed in one well cannot migrate into an adjacent well. See FIG. 2. The substrate can be affixed to the frame by pressure, e.g., with the use of a compliant gasket material, e.g., silicone or rubber.

It can also be affixed by gluing the frame to the substrate directly or by gluing the frame to a thin metallic substrate with holes where the wells are located and then gluing the thin metallic base to the substrate. The latter method has the advantage of providing a uniform grounding plane for the apparatus and reduces artifacts induced from the inherent resistivity of thin layers of metals such as that observed in vapor-deposited ITO.

Yet another method of manufacture involves applying a highly conductive ink to the ITO coated surface. This ink can be applied directly using standard printing devices or can be screen printed onto the surface. The pattern of the ink is set such that, when the upper well assembly is affixed to the ITO coated surface, the conductive ink is under the walls of each well produced by the upper assembly. A common electrode is attached to the upper frame so that it is in exclusive contact with the conductive ink, foil or conductive adhesive layer. This then forms a lower electrode assembly at each well and serves to ensure that the resistive change between one well and another well at any point across the device is constant.

The apparatus is designed such that a common ground electrode touches the conductive layer and thence to the lower ITO substrate (or the thin metallic layer under the frame which is in contact with the ITO layer). These electrodes provide a common connection to the ground plane of the lower substrate.

A series of upper electrodes are designed to match the number of wells within the apparatus. A series of electrodes for the field stimulation are also able to enter each well from the top of the device but these electrodes are positioned a short distance away from the ITO layer on the substrate such that there is no connection between these electrodes and the ones that are in contact with the ITO layer. Each of these upper electrodes can be connected to an electric field source independently or in parallel at varying numbers and patterns. In this way the user can examine the effects of many different stimulus conditions within a single multi-welled device.

The electrodes are attached to an electronic apparatus capable of creating an electrical pulse of the correct form. Pulses can be modulated for frequency, amplitude, shape (for example, sine, square or sawtooth) and number as well as time between pulse trains applied to the sample over a given period of stimulation exposure. The different wave forms can be generated with a number of electronic circuits or by the use of a function generator with suitable capabilities.

In order to allow adjustment to the height of each electrode above the substrate conductive surface, the electrodes consist of an electrode attached to a screw. The screw in turn is held in a plastic/metal housing that allows the height of the electrode to be varied by rotation of the screw. The electrodes are connected to the function generator or pulse circuit by means of binding posts to the planar upper electrode assembly.

Connections to the plates can also be accomplished where the electrodes are exposed on the upper side of lids that are commonly used on microtiter plates. A shelf unit (for example, within a standard Tissue Culture incubator, e.g., Thermo Cytomats) is able to hold these plates which are inserted from the front of the shelf. As the plate slides into the shelf it allows impact of connections from a pulse generator or function generator with the appropriate set of electrodes within the plate.

Thus the whole process can be automated thereby allowing for prolonged incubations. Under ideal TC conditions of temperature (37° C.), humidity (>80%) and CO₂ levels (typically 5%) the cells can be subjected to stimuli at appropriate levels and time intervals as discussed above.

Referring to the attached Drawings, FIG. 2 illustrates and exploded view of an example microtiter plate assembly. A transparent substrate (1) consisting of glass, polycarbonate or other suitable material is coated with a transparent conductive surface (2). This surface is ideally composed of indium tin oxide (“ITO”). A conductive grid (3) is printed or otherwise attached to the ITO surface such that the lines formed by this grid are eventually located under each of the walls of the upper well assembly (5) affixed to the assembly. The conductive grid can be formed by vapor deposition of a metal such as gold, silver, copper for example or by printing a conductive epoxy consisting of silver solids within an epoxy base or a suitable conductive ink.

Still referring to FIG. 1, the upper well assembly (5) is attached to the lower assembly (1-3) either using a silicon adhesive or through the use of a UV-curable adhesive or through the conductive epoxy forming the conductive grid (3). Another conductive layer (6) is affixed to the bottom of the substrate such that this forms a narrow perimeter around the external edge of the substrate. This conductive surface can also be vapor deposited gold or other conductive metal such as copper or may be an adhesive foil made of these materials. A conductive tape or metallic strip (copper or other metal) is affixed on the sides of the plate assembly (4) such that, upon assembly of the whole device this is conductively connected to the lower electrode grid (3) and to the lower electrode surface (6). Components 2, 3, 4 and 6 form the lower conductive electrode assembly.

Still referring to FIG. 1, the upper well assembly (5) is composed of a plastic or other non-conductive material (e.g., polystyrene, polycarbonate, polyolefin, polyethylene or may be any inert material such as a polyphenolic). Each well in the assembly may be round or square. The entire assembly may be of any dimension but preferably match with the dimensions for a microtiter plate as defined by the Society for Biomolecular Screening. In this geometry the number of wells can be varied from 6 to 1536 or higher. A 96-well version would have 8 wells×12 wells while a 384 well version would have 12 wells×24 wells. A 1536 plate would have 32×48 wells. As used herein, each well is a “cell retaining unit.”

While the geometry of the wells can be round or square in a microtiter plate they ideally are of equal spacing both in X and Y dimensions. A 96 well plate would have wells at 9 mm on center. A 384 well plate would have wells 4.5 mm on center and a 1536 well plate would be 2.25 mm on center. The height (or depth) of each well can be varied between, for example, 2 mm and 40 mm. Of course, other configurations may be readily obtained.

Referring to FIG. 1, in use the plate can be positioned onto a metallic and conductive surface such that the lower conductive layer (6) provides a conductive path between the lower metallic surface and the conductive grid (3) and this, in turn, provides a continual conductive path to the ITO layer (2) within each well. Consequently, if the lower metallic surface is held at electric ground potential (0 V) then the entire lower conductive surface will be held at the same potential. Furthermore, the resistance between any point in this conductive path and the ITO surface at the centre of any of the wells will be constant. The resistance depends on the amount of ITO deposited and can vary from a few milliohms to an infinite resistance if the ITO layer is omitted.

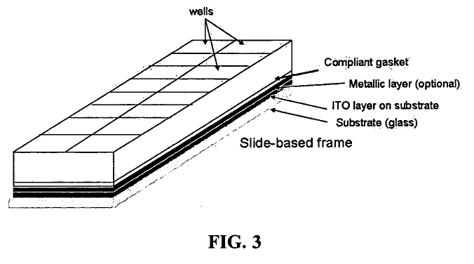

FIG. 3 illustrates an example microscope slide based system that uses an ITO coated microscope slide to effect the same principle. In this case each upper well assembly is 25 mm across by 75 mm long and may contain wells as above at 9 mm or 4.5 mm on center. The upper well assembly is again a molded polystyrene or polycarbonate. The well assembly may have a compliant silicone or rubber gasket attached such that the gasket is of the same size as the well walls. A metallic grid may be affixed directly to the lower gasket such that, when an ITO coated surface is held against the metallic surface the grid acts as a common connector between the entire surface.

The lower slide assembly may be held in place onto the upper well assembly and held tightly enough to the metallic conductor to effect a good electrical connection and to allow liquid to be held within each well. This can be achieved by gluing the assembly together using conductive epoxy or silicone adhesive or via pressure provided by means of plastic or metallic clips that provide pressure between the lower surface of the slide and the upper well assembly. Four of these devices can be inserted into a plastic frame that holds the assemblies side by side. This frame is of the same dimension as a microtiter plate. A conductive perimeter tape or metallic strip connects the electrode surface (for example, ITO) to the outside world. These conductors may be soldered or glued to the side of the device and connect through corresponding electrical connections in the frame such that when the frame is sitting on a conductive surface there is a conductive path to the ITO surface with low resistivity.

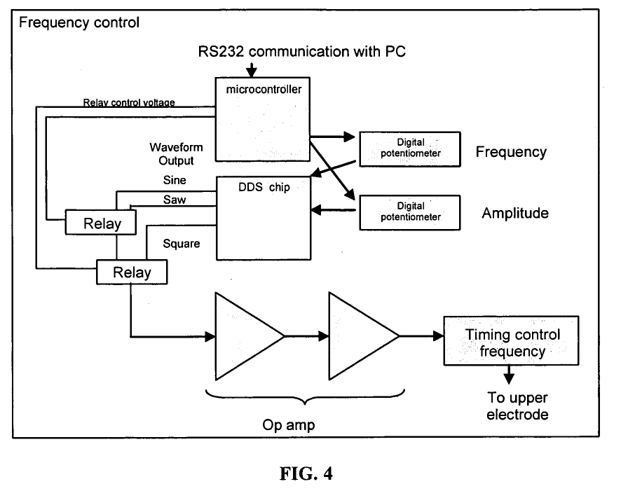

FIG. 4 illustrates a block diagram of frequency pulse stimulating circuitry. Values for pulse duration, pulse shape, frequency and amplitude are sent from the PC to the microcontroller. A program on the microcontroller adjusts the digital direct synthesizer (“DDS”) chip and operational amplifier (Op Amp) circuitry to provide the output needed. A separate circuit regulates how long the signal is applied to the upper electrode. The DDS chip can provide sine, square and sawtooth wave patterns that can be selected based on selection of the output pin from the DDS chip through the use of relays or switches controlled by the outputs from the microcontroller.

Frequency can be varied between 0 Hz and 1 MHz or higher (depending on the DDS used). The output from the device may be sent to the upper electrode assembly for the stimulation apparatus. Multiple stimulator circuits can be employed where each one provides a signal to a separate set of electrodes. In one embodiment, each row in a plate may be stimulated with a separate set of electrodes attached to a separate circuit set at different frequencies, amplitudes and wave shapes. In yet another embodiment each well receives a separate signal provided by a distinct circuit.

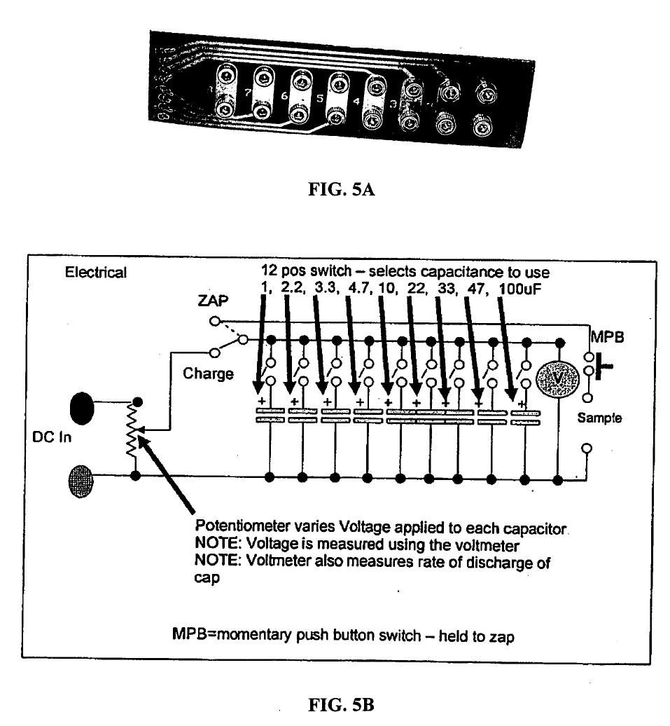

FIG. 5A illustrates an example upper electrode assembly for 16-well design (96-well plate compatible). Each row of two electrodes may be coupled to a distinct electrical circuit. The electrode assembly consists of a copper conductive layer etched to provide the electrical traces from an electrical connector to each set of electrodes to be stimulated in parallel from the same signal. This device can be modified to allow entire rows to be stimulated at one time or even the whole plate is stimulated by connecting all 96 (or 384 or 1536, among others) electrodes to a single input. The device shown allows varying stimulation effects to be analyzed and is valuable in selection of optimal criteria during assay development. In FIG. 5B, individual addressing of each electrode in the array can be achieved using a multi-position switch or could be achieved using a set of relays wherein each relay controls electrical connection of the pulse circuit to a single electrode of a subset of electrodes.

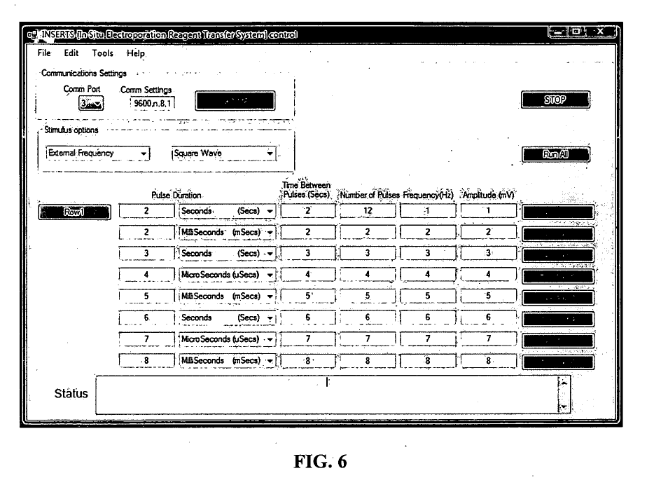

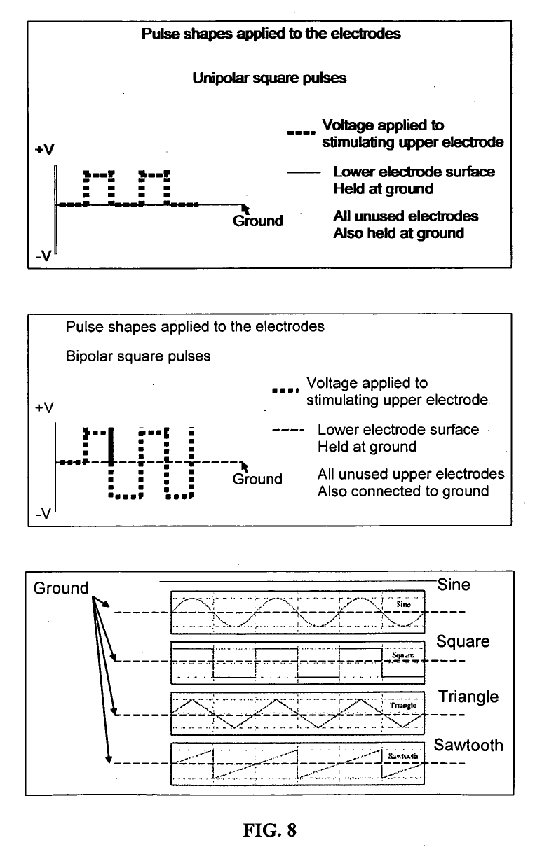

FIG. 6 depicts an example software interface for controlling the application of pulses to the microtiter plate system. Pulse durations between, for example, 2 microseconds and several minutes can be selected. Time between applied pulse trains can also be varied in this manner. The applied signal can be selected to be either DC or from the frequency circuit shown in FIG. 4. If the signal to be applied is a DC pulse the duration is set using the control circuit shown below. Magnitude of the pulse (amplitude) is also selectable from the graphical user interface (“GUI”) and, for a DC pulse, can be varied from 0 VDC to about 200 VDC through the microcontroller circuit and regulation of a voltage divider circuit. Applied frequencies (from the circuit shown in FIG. 4) can be adjusted between 0 Hz and about 1 MHz. Amplitudes can be varied between 0-20 V p-p. Application of these signals to the appropriate electrodes are regulated by the circuit shown as a block diagram in FIG. 8. Multiple pulse trains can be applied and time between pulse trains varied (typically between about 1 microsecond and several seconds).

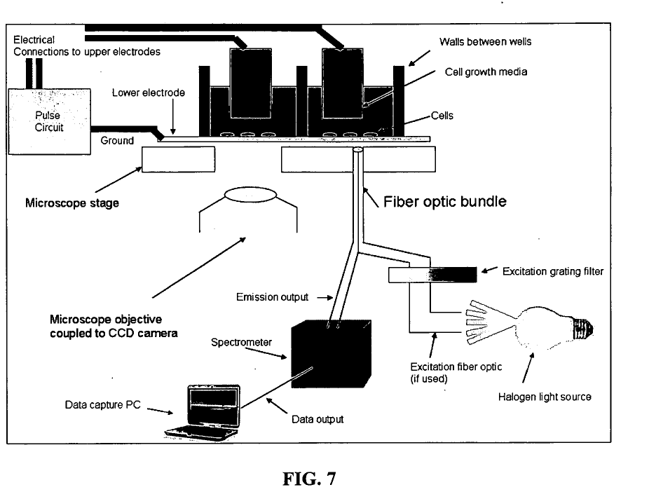

FIG. 7 includes a diagram of the setup for measurement of calcium flux from cells stimulated with electrical pulses (frequency stimulation). Stimulation of calcium flux in excitable cells (neurons or muscle cells) is driven by the application of electrical pulses to the cells growing as adherent cultures on the lower ITO electrode surface. To promote adhesion of the cells the ITO layer may further be coated with biological matrices known to promote cell adhesion (e.g., polylysine, fibronectin, collagen, matrigel, agarose, etc.). The pulse circuit (upper left; see FIG. 4) stimulates cells growing on the lower electrode surface of each well. Pulses of various shapes, frequency, amplitude can be applied.

Data can be collected either as images using the inverted microscope objective and attached CCD camera, or emitted signal can be captured using the fiber optic system installed in the lower stage of the microscope. For fluorescence measurements, one may stimulate using a halogen light assembly (Ocean Optics, FL) through a multiple fiber optic bundle (Ocean Optics). Excitation wavelength and bandpass is chosen using the variable wavelength exitation filter (Ocean optics) inserted in the optic path. Emitted fluorescence is captured in a separate optic fiber that is sent to a spectrometer (or photomultiplier tube) coupled through a USB interface to a personal computer (“PC”). Output is quantified using the software provided with the unit. To minimize the effects of extraneous light the entire assembly is contained within a light tight box. Luminescence measurements are made by monitoring the output in the emission fiber without the use of the excitation stimulus.

FIG. 8 illustrates pulse shapes that can be applied to the upper electrodes in the stimulation system, including the shape of unipolar pulses that can be applied to the upper electrodes. The lower ITO surface is held at ground potential. Pulses can be reversed so that they go below zero V. Pulses can also be applied where the signal varies both above and below zero V where the lower electrode surface is held at ground. Such voltage pulses may be symmetrical about the ground value as shown or may be unequally distributed. Additional pulse shapes that can be produced by the circuit shown in FIG. 4 and applied to the upper electrodes are shown below.

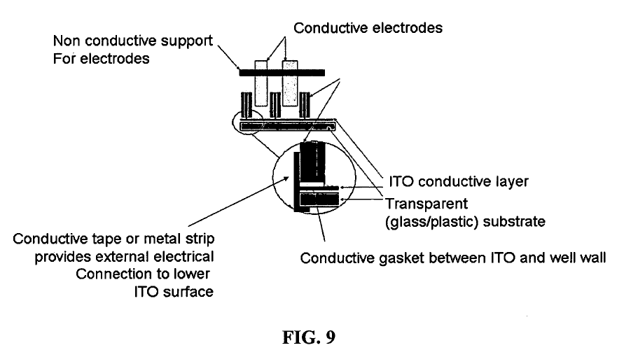

FIG. 9 is a side view of conductive plate, which shows two wells from the device in side profile with the upper electrodes inserted into the wells. The upper electrodes are metallic rods of a length that, when the non-conductive lid (support) is touching the upper lip of the wells, the lower surface of each electrode is held at between 0.2 mm and 5 mm above the ITO conductive surface. In one embodiment of the invention these electrodes are affixed to a screw through the plate lid and the height of each can be varied based on the position of the electrode on the screw. Each electrode is attached via a metal trace embedded into the non-conductive support to the stimulating circuitry. Each electrode can be attached separately to the stimulation control circuitry or multiple electrodes can be connected together (on the same trace).

As illustrated in FIG. 9, the upper electrodes could be encompassed as a part of the lid design for the plate where the electrodes are held in the plate throughout the experiment. Under these circumstances the electrical connections with the upper electrodes may be via spring loaded contacts that impact with the electric circuit comprising the connections to each set of electrodes (FIG. 5). Alternatively the electrodes could be inserted into the plate only when stimulating the cells. In this case electrical connections may be made directly to the electrode assembly PCB.

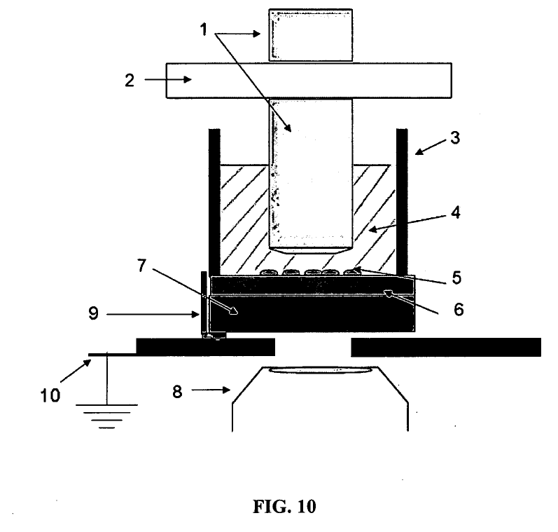

FIG. 10 is a side view of electrode assembly in use. The conductive upper electrode (1) extends through a non-conductive gasket (2) and into a well (3) of the microtiter plate assembly. A suitable medium for transfection or stimulation of the cells (4) is applied on top of cells growing as adherent monolayers (5) on the ITO conductive surface (6) applied to the glass or polycarbonate surface (7). To enable direct measurement of parameters such as myocyte length during contractions stimulated by the application of electric pulses to the cells or examination of calcium flux in the cells during stimulation of neuronal cells (for example), the cells can be visualized through an inverted microscope objective (8) during the application of the stimulus to each well.

Still referring to FIG. 10, the connection between the ground plane (10) and the lower ITO surface is provided through the connection between the metallic and conductive support (10) through the conductive layer (9) to the ITO surface and attached conductive grid. The plate can be moved both in X and Y direction relative to the microscope objective and this allows all wells of the plate to be visualized. The microscope objective could be a part of a high content screening automated system for visualization and measurement of cells in culture.

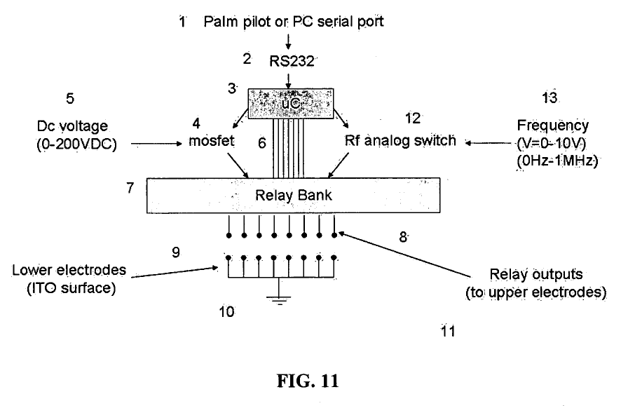

FIG. 11 depicts a circuit for controlling application of a signal to the upper electrodes. A microcontroller (3) communicates with the graphic user interface (GUI; FIG. 6) via, for example, RS232 (serial) communication. The GUI takes the user input for pulse duration and pulse type (frequency or DC voltages) and, based on these selections, instructs the microcontroller to activate a switch (12) that can allow high frequency pulses to pass through or a Mosfet transistor output (4) that allows a low voltage signal from the microcontroller to regulate the application of a DC voltage provided by an external power supply (5). Software running on the microcontroller regulates the output pin voltages. The output voltage on these IO pins in turn is used to activate the other electronic devices, e.g., the Mosfet power transistor (4), the RF analog switch (12) and the relays coupled to each of the upper electrode circuits (7). The frequency circuit (13) is a separate circuit and is the same as shown in FIG. 4. The lower electrodes are connected to ground.

The relays are SPDT CO (single pole double throw, closed/open) relays such that, without an applied voltage at the relay circuit the connections through the relays to each of the upper electrodes are at ground. When an IO pin from the microcontroller and connected to the input path of the SPDT CO relay is sent high (+5 VDC) this activates the relay and the switch connects the appropriate signal (DC or frequency) to the upper electrodes attached to the relay. The length of time a pulse is applied to the upper electrode is regulated by the time that the IO pin from the microcontroller connected to the gate of the Mosfet or to the input pin of the RF switch is held high. The microcontroller is able to provide a pulse as short as about 1 microsecond and can be held open indefinitely.

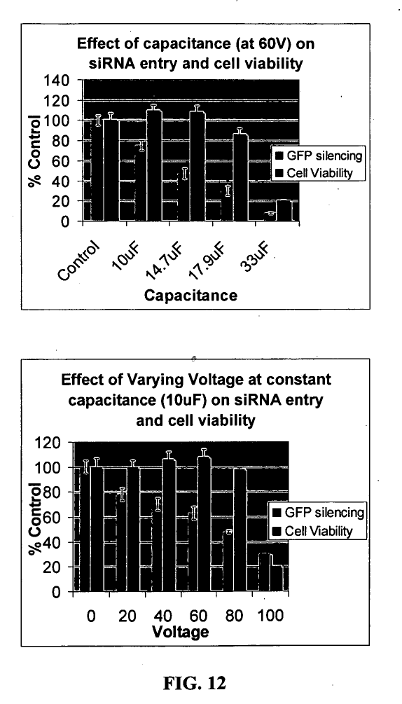

FIG. 12 illustrates use of DC pulses to obtain electroporation and sample uptake into cells using siRNA silencing of green fluorescent protein expression as an example. HeLa cells stably expressing green fluorescent protein (“GFP”) were cultured on electrically conductive ITO coated slides in the presence of an siRNA against GFP. Pulses with the indicated voltage and capacitance (higher capacitance equals longer pulse duration) were applied and the slides incubated at 37° C. in a CO₂ incubator for 24 h after pulsing. The amount of GFP present within the cells was monitored using a BMG Fluostar fluorescent plate reader adapted for holding the slides. The SD values plotted represent the standard deviation (“SD”) across 6 readings at various positions within each slide. Cell viability was subsequently monitored on the same slides by incubation with Cell Titer Blue (CTB; Promega, Madison, Wis.).

The conversion of CTB was used to calculate the percentage of viable cells on each slide using the BMG reader as above. It can be seen from FIG. 12 that uptake of the siRNA is dependent on applied voltage as well as the duration of the pulse (capacitance). At 60 V and <18 μF we can get good silencing without significant effects on cell viability. However, at certain values (>80 V; 10 μF or 60 V; >17.9 μF) the cells are killed by the application of the electric pulses.

We have adapted the methods shown in the figure above to the microcontroller based device shown in FIG. 11. Instead of varying capacitance to obtain the voltage and pulse duration needed, we can vary the pulse time and applied DC pulse voltage directly with the microcontroller circuit.

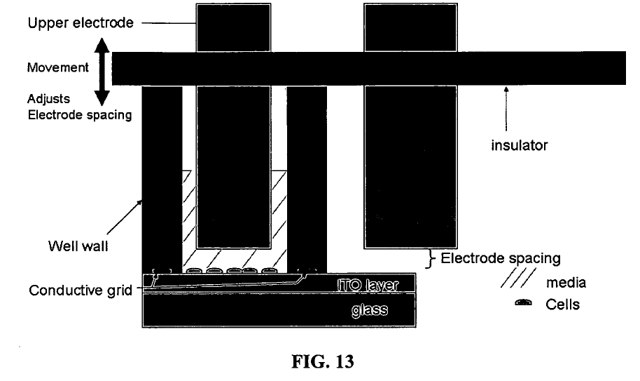

FIG. 13 illustrates movement of the upper electrode assembly in Z direction. The signal applied between the upper electrodes and the ITO surface can be varied in intensity by altering the amplitude of the applied pulses and can also be altered by varying the height of the electrodes above the ITO layer. The latter is achieved by means of a motor assembly attached to the upper electrode carrier.

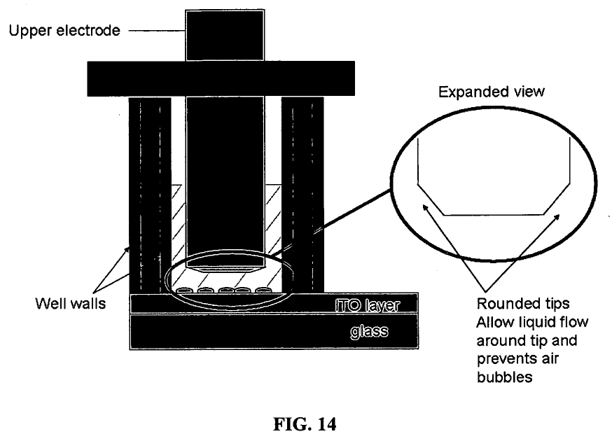

The electrode tips may optionally be rounded as shown in FIG. 14. This allows liquid flow around the tip and minimizes entrapment of air bubbles. Furthermore, the rounding effectively increases the distance between the electrode and the conductive grid printed on the ITO surface. Thus the electrical signal applied to the electrodes will take the path of least resistance. By rounding the electrodes we ensure that the distance between the lower surface of the upper electrode and the ITO is the shortest path.

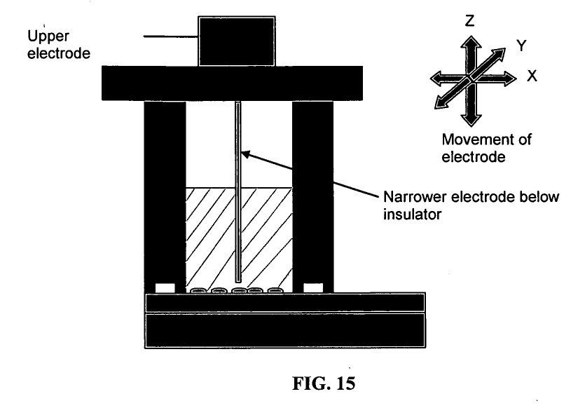

FIG. 15 shows a narrower upper electrode inserted into the well allows selective stimulation of discrete regions within each well. By moving the electrode assembly in X and Y we can position the electrode over the cell surface at discrete locations. Pulses can consequently be applied to select cells within the well. The cells may be visualized through the microscope setup shown in previous figures during the positioning of the electrodes. Using an HCS instrument it would be feasible to select discrete locations within a well where stimulation would be applied. These coordinates would be stored and used to position X and Y motors moving the upper electrode assembly over the right coordinates. Appropriate pulses would then be applied to each microelectrode separately.

This apparatus could examine the networks of communication between neuronal cells in culture or could be used to strengthen synapses in discrete regions and examine effects of this on additional cell behaviors in more remote locations within the well. Alternatively, in a mixed cell type culture system the effect on a second cell type can be studied in the presence of stimulation of a first cell type. The first cell type could be a nerve cell and the second cell type could be a muscle cell for example.

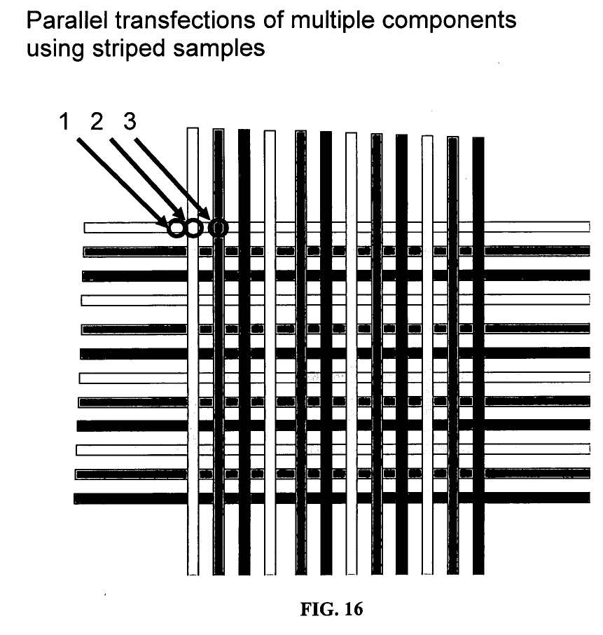

FIG. 16 illustrates an array-based transfection of molecules into cells. Since the invention may be used for electroporation of samples into cells cultured on top of them this extension of the invention allows measurement of the effects of multiple samples and the interactions between these when co-transfected into the cells. In this aspect, the samples to be studied are applied within a well (where the well can be of any size or shape) as stripes as shown in FIG. 16. Such stripes are of a width such that several cells may be attached across the width of the stripe. A discrete distance is allowed between each stripe to prevent the samples from intermixing on the surface except at junction points (2 and 3).

In the example shown in FIG. 16, stripes represent different samples applied to the surface of the conductive plate. Cells are dispensed into the well and allowed to adhere over top of the striped surface. By using sufficient numbers of cells and a suitable number of stripes with appropriate dimensions (width), a relatively uniform covering of cells across the surface of the striped array is obtained. At this point an electrode is introduced into the well. The electrode geometry is ideally of a size to allow coverage over the entire array surface to be studied. By applying a suitable pulse between the upper electrode and the lower ITO surface, the cells growing on top of the surface can be electroporated allowing the entry of the samples into the cells.

The samples could be siRNAs, miRNAs, DNAs, peptides, proteins, sugars, small molecules such as drugs or chemical compounds. In an example embodiment, the samples are RNAs (either siRNAs, miRNAs (mimics or inhibitors), piRNAs) or DNAs (e.g., cDNA plasmids)). These reagents may be held in location during the application of the cells by means of a polymer surface applied to the top of the ITO coating. Such polymers may include PEI, polylysine, poly-D-lysine, polymeric peptides consisting of histidine and lysine or modified PEG or DOTAP. Cationic peptides may be preferred but the surface modification should have the following features; it should be able to hold and stabilize the molecules printed on the surface, the molecules must be held when the cells or media are applied to the well and yet must be released upon application of a suitable electrical signal. PEI has previously been demonstrated to have all of these properties for delivery of DNA (Yamauchi et al., 2005).

The samples can be applied to the surface using capillary quill tips used for microarray manufacture, using various other spotting devices such as ink jet printing or via application using a draughtsman's pen or even a fine needle syringe dispenser. Upon application of the samples as stripes there are discrete regions that are visible from the pattern. Still referring to FIG. 16, in region (1) is a single layer of a single sample. Cells electroporated over top of this region would only be exposed to this one sample at a 1× concentration. In contrast, region (2) in FIG. 16 is where a horizontal stripe and a vertical stripe intersect. Because, in this case, both stripes are of the same material the result will be that cells growing immediately over this region and exposed to an electroporation pulse will be exposed to a single sample but at 2× concentration relative to that seen in position (1). Where a horizontal stripe and a vertical stripe are of different samples (3), the result for cells growing on top of this location will be that, upon electroporation, the cells in this region are exposed to two distinct samples, each at 1× relative concentration. The advantage of this striping approach is that multiple regions across the striped surface have regions of overlap of two samples. Therefore, the activity of the combination of the two samples can be validated in replicates even within each array.

It is feasible that the samples could be combinations of RNA and DNA reagents. In this way, a plasmid for a gene such as green fluorescent protein could be in one stripe and an siRNA against another gene be in a second sample. Where stripes overlap, the amount of the plasmid expression (GFP signal in cells subsequently growing over this spot) would be an indication of transfection efficiency. The degree of effect produced by the siRNA overlapping with the plasmid stripe could then be quantified by examining key morphological/phenotypic or even genomic changes in cells over this region.

Alternatively where the 2 stripes are each a different microRNA mimic (for example), the combination of these two reagents on a phenotype change in the cells can be monitored. Since multiple stripes can be made in X and Y direction and the size of the area to be examined is not limiting it is feasible that an array of this nature could examine all of the siRNAs against all genes within a genome. Currently there are 7700 genes in the “druggable genome” for human genes (ion channels, receptors and enzymes) for example. If each stripe has a width of 10 μm and a 5 μm spacing between each one then an array of all “druggable genes” striped would be ˜11.5 cm×11.5 cm. A tray of this dimensions (with a glass or plastic substrate with an ITO coating) can be made and an upper electrode of the same dimensions also made to fit the tray and be located close to the ITO surface at the bottom of the tray.

Within each well of a 96-well plate the well dimensions are 6 mm×6 mm. If each stripe is 20 μm and space between stripes is the same then this would suggest that 150 stripes could be supported in each direction within a single well. 96 wells could be used and striped independently. Therefore, a single plate could be used to interrogate complex interactions between up to 150 of the same molecules within each well. In this case the cells within each well can also be exposed to other external factors (growth factors, small molecule compounds, peptides, proteins, etc.) and the sensitivity of the cell physiology to the combination treatments examined with this third parameter as well.

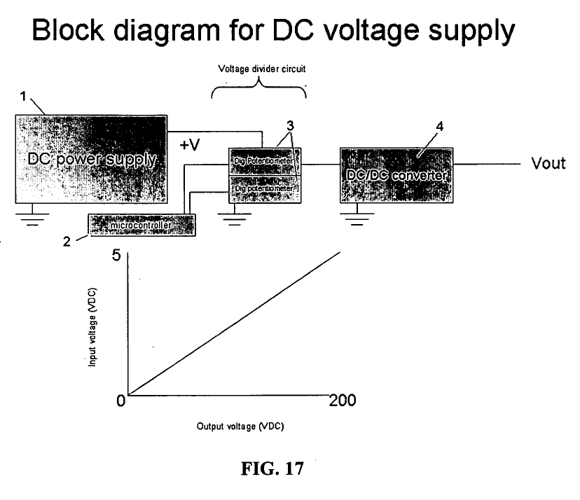

FIG. 17 illustrates a DC pulse supply block diagram. The DC pulse supply uses a DC power supply (1) to provide a constant voltage to 2 digital potentiometers (3) that are configured as a voltage divider circuit. The values for the digital potentiometers are adjusted using a microcontroller (2), that communicates with the control PC, for example, via RS232. The output voltage from the voltage dividers can be adjusted between 0 and 12 VDC. This in turn is fed into a DC/DC converter (4) that outputs a larger voltage signal directly proportional to the input voltage. Such a DC converter is supplied by EMCO. The output voltage to input voltage relationship is shown in the bottom of FIG. 17. The output from this circuit is fed to the timing circuitry (shown in FIG. 11) where another microcontroller regulates the time of the applied pulse through the opening of a MOSFET transistor (FIG. 11) allowing the DC signal to pass for the time specified by the time that the IO pin on the microcontroller is held high. The DC signal output from the Mosfet is sent directly to a subset of the upper electrodes for the device.

### ADDITIONAL EMBODIMENTS

The subject matter of the present disclosure may take the form of several different embodiments, including the following non-limiting examples.

An apparatus for use in electronic stimulation of cells may include a first electrode having a plurality of cell retaining units; and a second electrode having a plurality of electrically conductive leads complementary to the plurality of cell retaining units; wherein the second electrode is moveable into engagement with an electronically conductive cell culture medium to thereby provide a closed electronic circuit between the first electrode and the second electrode, and the electronic circuit provides substantially homogenous electronic stimulation within each cell retaining unit.

Each of the plurality of electrically conductive leads may be located within the center of the complementary cell retaining units upon engagement. Likewise, the plurality of cell retaining units may each comprise a surface upon which cells are capable of adhering, each of the plurality of electrically conductive leads being located within the center of the complementary cell retaining units upon engagement, and each electrically conductive lead being equidistant to all points on the complementary surface upon which cells are capable of adhering.

In another embodiment, the number of electrically conductive leads of the second electrode is equal to the number of cell retaining units of the first electrode. In yet another embodiment, the number of electrically conductive leads of the second electrode is greater than the number of cell retaining units of the first electrode. In a further embodiment, the number of electrically conductive leads of the second electrode is twice the number of cell retaining units of the first electrode. Each of the plurality of electrically conductive leads may be electronically isolated from the other electrically conductive leads and is independently configured to provide a unique electronic circuit.

Furthermore, the apparatus may include an electric current generator, which is capable of producing an constant or variable electronic current. The variable electronic current may vary in amplitude, duration, or frequency with respect to time. Similarly, the variable electronic current may vary according to a configurable predetermined program.

In another embodiment, the plurality of cell retaining units are optically transparent. For example, the plurality of cell retaining units may be constructed from glass or polycarbonate.

In still another embodiment, the plurality of cell retaining units are electrically conductive. For example, the plurality of cell retaining units may have a surface that is at least partially coated with indium tin oxide. Moreover, the first electrode and the second electrode may be constructed from non-oxidative materials, e.g., the second electrode may be at least partially constructed from gold, silver, copper, or an alloy thereof.

In another embodiment, the plurality of cell retaining units each comprise a surface upon which cells are capable of adhering. For example, the cell retaining units may be coated with a polymer that facilitates cellular adhesion, such as polylysine.

In a typical embodiment, the plurality of cell retaining units is in the form of a multi-well plate.

In another embodiment, a method of electronically stimulating cells include providing a first electrode having a plurality of cell retaining units; providing a second electrode having a plurality of electrically conductive leads complementary to the plurality of cell retaining units; culturing cells within one or more of the cell retaining units, wherein the cells are adherent on a surface thereof and the cells are in an electrically conductive cell culture medium; moving the first electrode into engagement with the second electrode to thereby complete a closed electronic circuit; and applying an electronic current through the closed electronic circuit, wherein all of the cells within each cell retaining unit are exposed to substantially the same electronic field.

In the foregoing method, the cell culture medium may include an exogenous agent and the applied electronic current induces transient pores in the membranes of the cultured cells sufficient to permit the exogenous agent to enter the interior of at least of portion of the cultured cells. For example, the duration of the electric current may be less than about 100 microseconds, and the exogenous agent may include a polynucleotide, a RNA molecule or DNA molecule, a plasmid, a siRNA molecule, a polypeptide, or an antibody. Similarly, the exogenous agent causes a physiologic change in the cells. That is, the exogenous agent may include a drug candidate.

The cultured cells may include unicellular microorganisms or they may include cells from a tissue of a multicellular organism. Additionally, the electronic current may mimic the endogenous electronic stimulation encountered by the tissue in the multicellular organism. For example, the electronic may be continuously applied.

In yet another embodiment, a multi-well plate includes an optically transparent base having a surface coated with an optically transparent electric conductor; and a plurality of non-electrically conducting walls attached to and extending vertically from the grid at their proximal ends, wherein the plurality of walls and the base form a plurality of wells, each having an opening formed by the distal ends of the walls. The multi-well plate may also include an electrically conductive strip applied to the perimeter walls, extending from the base to the distal ends of the perimeter walls. Additionally, a multi-well plate may include an electrically conductive grid disposed between the walls and the base. In an example configuration, the transparent electric conductor includes a layer of indium tin oxide. Typically, the plurality of wells comprise 96 wells arranged in a 12 by 8 pattern, 384 wells arranged in a 12 by 24 pattern, or 1536 wells arranged in a 32 by 48 pattern.

In still yet another embodiment, an electrically conductive array includes multiple electrodes complementary to multiple cell retaining units; each of the multiple electrodes including a plurality of electrically conductive leads complementary to each cell retaining unit. The array may also include a second electrode moveable into engagement with an electronically conductive cell culture medium to thereby provide a closed electronic circuit. Also, the plurality of electrically conductive leads can be inserted into a cell retaining unit simultaneously, e.g., so that the distance of the plurality of electrode leads from the second electrode can be adjusted, i.e., the plurality of electrode leads can be adjusted in three dimensions within each cell retaining unit.

The subject matter hereof may also be embodied as a multiwelled (plate) apparatus including an upper assembly defining individual sample wells able to hold biological reagents within each well, including a multiwelled plate where the specifications for the plate dimensions are identical to those provided by the Society for Biomolecular Screening (http://www.sbsonline.org/msdc/approved.php and documents therein). For example, a multiwelled plate may have wells that are 9 mm on centre and are in an 8×12 arrangement (coinciding with the well spacings of a 96-well microtiter plate). Additionally, a multiwelled plate may have wells that are 4.5 mm centre to centre in a 16×24 arrangement (coinciding with the well spacing of a 384 well microtiter plate). A multiwelled arrangement may have spacing between wells of less than 4.5 mm, wherein the wells are in an X by Y arrangement where X and Y can be any number.

A lower surface for the wells may include glass, polystyrene, polycarbonate or other transparent surface. A coating may be applied to the lower surface that is transparent but electrically conductive (e.g., indium tin oxide (“ITO”)), which functions as a lower electrode surface within each well.

Yet another embodiment includes means for ensuring a uniform electric field between upper and lower electrodes, as well as means for connection of the lower electrode as close as possible to each well, wherein connections to the lower electrode are evenly spaced around the perimeter of the well.

Yet another embodiment include an assembly of metal rods (upper electrodes) with spacing corresponding to those between wells of the multi-welled device and whose dimensions allow entry into each well of the device and yet whose dimensions are as close to those of the well to ensure an even electrical field between these electrodes and the lower well surface. The lower surface of each of these electrodes may be in the same horizontal plane.

Still yet another embodiment includes means for altering the height of the upper electrodes from the lower electrode surface, as well as a method where the upper electrode assembly is moved by means of a motor(s).

Another embodiment includes a single electrode that is much smaller than the well it is inserted into such that it can be inserted into each well and its position can be adjusted in X, Y and Z dimensions either manually or by means of motorized axes. This electrode may have dimensions that are much smaller than those of each of the wells into which it is inserted allowing electric pulses to be applied between it and the lower electrode surface in multiple discrete locations within each well.

The electrodes may be of a significantly smaller diameter than the well and multiple electrodes can be inserted into each well at the same time. Accordingly, individual cell locations within a well can be identified, the electrode placed above such cells and used to selectively electroporate subpopulations of cells within a single well. An array (X by Y) of electrodes may be inserted into each well at the same time together with a means whereby either all electrodes are stimulated at the same time with an electrical pulse or where each electrode is stimulated independently. All electrodes may be utilized at one time to conduct an electrical pulse between the upper electrodes and the lower electrode surface. In another embodiment, each electrode in the array can be addressed separately by means of a relay or switch and where the electrical pulse applied to each electrodes can be varied in amplitude and duration. Likewise, the electrodes to be used can be selected and pulse duration, amplitude and distance between upper and lower electrodes are controlled by means of a computer.

In an typical embodiment, cells are grown on the lower conductive surface of the multi-welled device. Application of an electrical pulse between the upper and lower electrode assemblies allows electroporation of the cells on the lower electrode assembly and passage of exogenous materials into the cells. The design of the upper electrode minimizes the potential for air bubbles to be trapped between the upper and lower electrodes when the upper electrodes are lowered into the medium within a well for electroporation.

Cells can be monitored for uptake of materials using a microscope to visualize fluorescently tagged molecules or where the cell responds either by increasing a signal (e.g., luminescence based reporter gene or fluorescent reporter expression) or decreases expression of these same signals through silencing of genes linked to these reporters. A microscope can be automated such as with an automated high content screening system.

In another embodiment, the lower conductive surface may be coated with a material that allows a biological sample to be located at this defined position while cells are growing on the surface. Samples may be introduced into cells are placed on the lower conductive surface ahead of the cells. Such a surface may be charged (e.g., coated with polylysine) and the sample to be used in oppositely charged (e.g., RNA, siRNAs or DNA) preventing dissolution and migration of samples from their location.

Another embodiment hereof takes the form of an open array, as well as a method wherein samples are located at discrete positions within each well, i.e., an array of samples within each well where multiple samples are deposited as discrete spots within each well. Sample may be “striped” onto the surface of the well in either an X or a Y direction such that samples produce multiple stripes in either direction and overlay at the intersections between the X and Y directions. In this way electroporation of cells immediately above the samples will allow entry of either a single sample (no overlap) or cotransfection of multiple samples (at the XY intersections).

Yet another embodiment includes a method for consistent electroporation in non-adherent (suspension) cells whereby cells in suspension media are dispensed into each well of the multi-welled electroporation plate, the plate(s) are placed on a standard microtiter plate centrifuge (to sediment the cells onto the base of the plate, i.e., the lower electrode), the plate(s) are removed and placed into the electroporation apparatus and the upper electrodes are lowered to a set distance from the lower electrode. An electric pulse of a voltage and duration sufficient to permeabilise the majority of the cells without resulting in significant cell death is applied across the upper and lower electrodes in the presence of the reagent to be transfected. The reagent gains access to the interior of the cells. Since the cells are sedimented the gap between the electrodes can be reduced and this lowers the voltage required for electroporation. In turn, since all of the cells are nominally within the same plane (near the bottom of the well) all of the cells will experience a similar electric field and this will increase the reproducibility of the uptake across all of the cells within a well.

The following illustrative explanations of the Figures and related Examples are provided to facilitate understanding of certain terms used frequently herein, particularly in the Examples. The explanations are provided as a convenience and are not limitative of the invention.

### EXAMPLES

Optimization of electroporation conditions for transfection of siRNAs in the plate-based format, comparison with current slide-based results. Using the electroplates, together with the electrode assembly described above, the optimal conditions for electrically induced permeabilization of the cells while maintaining the highest degree of cell viability were determined. Preliminary results from experiments that were performed with in situ electroporation of cells attached to the same electrically conductive surface on a slide-based system indicate that we can obtain permeabilization and entry of siRNAs into cells with voltages around 40-60 V and very short pulse durations (see FIG. 3). The initial experiments utilized HeLa-eGFP cells (stably expressing green fluorescent protein (“GFP”)) and were transfected with siRNAs specifically targeting the GFP protein expression. Specificity of the observed effects were validated by comparison with the effect produced using a scrambled siRNA sequence that should not produce silencing of GFP in the same cells.

Briefly, HeLa GFP cells maintained in culture were trypsinized from their flasks and counted before dilution into growth medium and dispensed into the plates. The cells were seeded in the wells of the electroplates and given time to adhere to the surface (lower electrode). After 24 h, the growth medium was removed, the cells washed briefly with buffer and buffer containing the siRNA of interest was added to the appropriate wells. The electrode assembly was inserted and the cells exposed to electrical pulses varying in number, intensity and duration. Cells were given a short time to take up the siRNA (2-15 mins) before exchanging the electroporation buffer for growth medium.

The plates were placed back into a CO₂ incubator and incubated for a defined time (24-72 h). At these times, the amount of silencing produced by the siRNA taken up into the cells was measured using a BMG Fluostar fluorescent plate reader to measure excitation at 475 nm and emission at 530 nm (excitation and emission wavelengths for GFP). After fluorescent measurements were made to determine degree of GFP silencing, the number of viable cells was determined using Cell Titer Blue (CTB; Promega, Wis.). Cell Titer Blue provided a homogeneous, fluorescent method for monitoring cell viability. The assay was based on the ability of living cells to convert a redox dye (resazurin) into a fluorescent end product (resorufin). Nonviable cells rapidly lost metabolic capacity and thus do not generate a fluorescent signal.

By comparison of the ratio of GFP silencing with the proportion of viable cells, the conditions were determined that allow uptake of the siRNA into the cells while minimizing cell killing. Parameters monitored during assay optimization included concentration of siRNA in the external medium; length of pulse duration; number of pulses given; intensity of the pulses; and number of cells within each well.

The siRNA arrays were spotted onto conductive glass slides using a Radius 3XVP arrayer. The 3XVP arrayer is also capable of arraying into the wells of microtiter plates. siRNA against GFP was used as for the experiments in welled plates however, the siRNAs were labeled with rhodamine dye so that the location of the dye can be readily observed using a standard fluorescence microscope. Initial experiments examined a number of reagents at differing concentrations to explore whether the mixture can hold the labeled siRNA samples in location when a solution was applied above the spots.

While a standard fluorescent microscope equipped with a CCD camera can record the events at a global scale within the region of a spot, the approach would benefit from access to some of the tools such as high content cell imaging currently being employed in high throughput screening labs. Vendors such as GE Healthcare (Piscataway, N.J.) supply instrumentation such as the Incell 1000 and Incell 3000 platforms that can be used for sub-cellular imaging and quantitation. Software algorithms for image analysis are now capable of evaluating images from these automated microscopic imaging systems and quantifying a cellular response based on these images. With 300 μm spots and a typical cell (such as HeLa cells), there are 10's of cells per spot. Image analysis algorithms can be used to quantify effects in each of the cells within the viewable area and determine outcome on various morphological or phenotypic outcomes from exposure to the siRNA species. Large arrays of siRNAs can thus be evaluated using this platform and this will further extend the utility of the platform into the drug discovery arena.

The foregoing description of some specific embodiments provides sufficient information that others can, by applying current knowledge, readily modify or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. In the drawings and the description, there have been disclosed exemplary embodiments and, although specific terms may have been employed, they are unless otherwise stated used in a generic and descriptive sense only and not for purposes of limitation, the scope of the claims therefore not being so limited. Moreover, one skilled in the art will appreciate that certain steps of the methods discussed herein may be sequenced in alternative order or steps may be combined. Therefore, it is intended that the appended claims not be limited to the particular embodiment disclosed herein.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims; or in the text itself, and, each of these documents or references (“herein-cited references”), as well as each document or reference cited in each of the herein-cited references (including any manufacturer's specifications, instructions, etc.), is hereby expressly incorporated herein by reference.

### REFERENCES

Citation of any references herein is not intended as an admission that such references are prior art or that they are necessarily material to patentability. All statements as to the date or representation as to the contents of these documents is based on the information available to the applicant and does not constitute any admission as to the correctness of the dates or contents of these documents. The following references are illustrative of the current state of the art as it is known to the inventors.
1. Yamauchi F, Kato, K. And Iwata, H. “Layer-by-Layer Assembly of Poly(ethyleneimine) and Plasmid DNA onto Transparent Indium-Tin Oxide Electrodes for Temporally and Spatially Specific Gene Transfer” Langmuir 21; 8360-8367.
2. Zhao, M, Bai, H., Wang, E., Forrester, J., and McCaig, C. J. Cell Sci. (2004) 117; 397-405 “Electrical stimulation directly induces pre-angiogenic responses in vascular endothelial cells by signaling through VEGF receptors.”
3. Qiu, Q., Sayer, M., Kawaja, M., Shen, X. and Davies, J E (1998) J. Biomedical mater. Res. 42; 117-127 “Attachment, morphology and protein expression of rat marrow stromal cells cultured on charged substrate surfaces.”
4. Berger, H J., et al (1994) Am. J. physiol. 1; H341-349 “Continual electric field stimulation preserves contractile function of adult ventricular myocytes in primary culture.”
5. Holt, E., Lunde, P K., Sejerstad, O M., and Chrsitensen, G. (1997) 92; 289-298 Basic Res. In Cardiology “Electrical stimulation of adult rat cardiomyocytes in culture improves contractile properties and is associated with altered calcium handling.”
6. Janigro et al., (2006) Biomed central Cancer 6; 72 “Alternating current electrical stimulation enhanced chemotherapy: a novel strategy to bypass multidrug resistance in tumor cells.”
7. Ircher, I. and Hood, D A (2004) J. Appl. Physiol. 97; 2207-2213 “Regulation of Egr-1, SRF and Sp1 mRNA expression in contracting skeletal muscle cells.”
8. Xia, Y., Buja, M., and Mcmillan, J B. (1998) J. Biol Chem 273; 12593-12598 “Activation of the Cytochrome c Gene by electrical stimulation in neonatal rat cardiac myocytes.”
9. Radisic et al. (2004) PNAS USA 101; 18129-18134 “Functional assembly of engineered myocardium by electrical stimulation of cardiac myocytes cultured on scaffolds.”
10. Sauer et al., (2002) J. Cell Sci., 115; 3265-3273 “The Dc electrical field induced calcium response and growth stimulation of multicellular tumor spheroids are nediated by ATP release and purinergic receptor stimulation.”
11. Wartenburg et al., (1997) Am. J. physiol. 272; R1677-1683 “Electrical fields enhance growth of cancer spheroids by reactive oxygen species and intracellular calcium.”
12. Balkowiec, A and Katz, D M (2002) J. Neurosci. 22(23); 10399-10407. “Cellular mechanisms regulating activity-dependent release of native Brain Derived Neurotrophic Factor (BDNF) from hippocampal neurons.”
13. Fields, R D, Neale, E A., and Nelson, P G (1990) J. Neuroscience 10(9); 2950-2964 “Effects of patterned electrical activity on neurite outgrowth from mouse sensory neurons.”
14. Dube, J. et al., “External electric fields induce morphological changes on human skin cells cultured in vitro.”
15. Reiher et al., (2005) App. Physics Letts. 86; 103901 “In Vitro stimulation of neurons by a planar Ti—Au-electrode surface.”
16. Raptis, L et al., (1995) Mol. Biotechnol. 4; 129-138“Applications of electroporation of adherent cells in situ, on a partly conductive slide.”
17. Raptis, L. et al., (1998) Promega Notes 67; 12“Inhibition of EGF-Induced ERK1 and ERK2 enzyme activation in NIH3T3 cell by ins situ electroporation of a nonpermeant inhibitor.”
18. Wegener, J., Keese, C R., and Giaver, I. (2002) Biotechniques 33; 348-357 “Recovery of adherent cells after in situ electroporation monitored electrically.”
19. Takata, K., Takahashi, Y., Mtsuzaki, T., Tajika, Y., Suzuki, T., Aoki, T. and Hagiwara, H. (2003) Acta Histochem. Cytochem 36(4); 317-323 “A simple electroporation method for the introduction of plasmids into cells cultured on coverslips for histochemical examination.”
20. Weaver, J C (1995) Methods Mol. Biol. 55; 3-28“Electroporation theory. Concepts and mechanisms.”
21. Ramos, C. and Teissie, J. (2000) Biochimie. 82; 511-518“Electroporation: a biophysical modification of cell membrane and a mechanism in exocytosis.”
22. Yang, T A et al., (1995) Nuc Acid Res. 23; 2803-2810“Efficient in situ electroportaion of mammalian cells grown on microporous membrane.”
23. Antov, Y., Barbul, A., Mantsur, H. and Korenstein, R. (2005) Biophys. Journal 88; 2206-2223 “Electroendosytosis: exposure of cells to pulsed low electric fields enhances adsorption and uptake of macromolecules.”
24. Kimura, K., Yanagida, Y., Haruyama, T., Kobatake, E and Aizawa, M (1998) Med Biol. Eng. Comput. 36(4); 493-498 “Electrically induced neurite outgrowth of PC12 cells on the electrode surface.”
25. Fire A., et al., (1998) “Potent and specific genetic interference by a double stranded RNA in Caenorhabditis elegans” Nature 391; 806-811.
26. Huppi, K., Martin, S. E., and Caplen, N. (2005) “Defining and assaying RNAi in mammalian cells” Molecular Cell 17; 1-10.
27. Raptis, L. and Firth, K. L. (1990). “Electroporation of adherent cells in situ.” DNA and Cell Biology 9:615-621.
28. Brownell, H. L., N. Lydon, E. Schaefer, T. M. Roberts, and L. Raptis. (1998). “Inhibition of Epidermal Growth Factor-mediated ERK1/2 activation by in situ electroporation of nonpermeant [(alkylamino)methyl]acrylophenone derivatives.” DNA and Cell Biology, 17:265-274.
29. Raptis, L., K. L. Firth, H. L. Brownell, A. Todd, W. C. Simon, B. M. Bennett, and M. Zannis-Hadjopoulos. (1995). “Electroporation of adherent cells in situ for the introduction of nonpermeant molecules” J. A. Nickoloff (ed.), Methods in Molecular Biology. Protocols for electroporation and electrofusion of Plant and Animal Cells. The Humana Press Inc., Totowa, N.J., Chapter 7:93-113.
30. Gambarotta, G., C. Boccaccio, S. Giordano, M. Ando, M. C. Stella, and P. M. Comoglio. (1996). “Ets up-regulates met transcription”, Oncogene 13:1911-1917.
31. Brownell, H., Firth, K. L. Kawauchi K., Delovitch T., and Raptis L. (1997). “A novel technique for the measurement of Ras activity: Electroporation of [□32P]GTP.” DNA and Cell Biology, 16:103-110.
32. Haruta, T., A. J. Morris, P. Vollevweider, J. Nelson, D. W. Rose, M. Mueckler, and J. M. Olefsky. (1998). “Ligand-independent Glut4 translocation induced by Guanosine 5′-O-(3-Thiophosphate) involves Tyrosine phosphorylation.” Endocrinology 139:358-364.
33. Firth, K. L., H. L. Brownell, and L. Raptis. (1997). “An improved procedure for electroporation of peptides into adherent cells in situ.” Biotechniques, 23:644-645.
34. Giorgetti-Peraldi, S., E. Ottinger, G. Wolf, B. Ye, T. R. Burke, and S. E. Shoelson. (1997). “Cellular effects of phosphotyrosine-binding domain inhibitors on insulin receptor signalling and trafficking.” Mol. Cell. Biol. 17:1180-1188.
35. Marais, R., Spooner, R A, Stribbling, S M, Light, Y., Martin, J. and Springer, C J. (1997). “A cell surface tethered enzyme improves efficiency in gene-directed enzyme prodrug therapy.” Nature Biotechnology, 15:1373-1377.
36. Boccaccio, C., Ando, M., Tamagnone, L., Bardelli, A., Michielli, P., Battistini, C. and Comoglio, P. M. (1998). “Induction of epithelial tubules by growth factor HGF depends on the STAT pathway.” Nature, 391:285-288.
37. Evans, D M, Azorsa, D A and Mousses, S. (2005) Drug Discovery Today:Technologies (in press).
38. Zhang, J. et al., (1999) J. Biomol. Screen. 4; 67-73.
39. Mousses, S., et al., (2003) “RNAi microarray analysis in cultured mammalian cells” Genome Res. 13; 2341-2347.
40. Erfle H, Simpson J C, Bastiaens P I, Pepperkok R. (2004) “siRNA cell arrays for high-content screening microscopy.” Biotechniques. 37(3):454-458.
41. Conrad C, Erfle H, Wamat P, Daigle N, Lorch T, Ellenberg J, Pepperkok R, Eils R. (2004) “Automatic identification of subcellular phenotypes on human cell arrays.” Genome Res. 14(6):1130-6.
42. Ma Z, Zhang J, Alber S, Dileo J, Negishi Y, Stolz D, Watkins S, Huang L, Pitt B, Li S. (2002) Lipid mediated delivery of oligonucleotide to pulmonary endothelium. Am J Respir Cell Mol Biol 27: 151-159.
43. Wilson, A., Zhou, W., Champion, H., Alber, S., Tang Z.-L., Kennel, S., Watkins, S., Huang, L., Pitt, B., and Li, S. Targeted delivery of oligodeoxynucleotides to mouse lung endothelial cells in vitro and in vivo. Molecular Therapy 12: 510-518, 2005

What is claimed is:

1. An apparatus for use in electronic stimulation of cells comprising
a first electrode having a plurality of cell retaining units; and
a second electrode having a plurality of electrically conductive leads complementary to the plurality of cell retaining units;
wherein the second electrode is moveable into engagement with an electronically conductive cell culture medium to thereby provide a closed electronic circuit between the first electrode and the second electrode, and the electronic circuit provides substantially homogenous electronic stimulation within each cell retaining unit.

2. The apparatus according to claim 1, wherein each of the plurality of electrically conductive leads is located within the center of the complementary cell retaining units upon engagement.

3. The apparatus according to claim 1, wherein the plurality of cell retaining units each comprise a surface upon which cells are capable of adhering, each of the plurality of electrically conductive leads is located within the center of the complementary cell retaining units upon engagement, and each electrically conductive lead is equidistant to all points on the complementary surface upon which cells are capable of adhering.

4. The apparatus according to claim 1, wherein the number of electrically conductive leads of the second electrode is equal to the number of cell retaining units of the first electrode.

5. The apparatus according to claim 1, wherein the number of electrically conductive leads of the second electrode is greater than the number of cell retaining units of the first electrode.

6. The apparatus according to claim 1, wherein the number of electrically conductive leads of the second electrode is twice the number of cell retaining units of the first electrode.

7. The apparatus according to claim 1, wherein each of the plurality of electrically conductive leads is electronically isolated from the other electrically conductive leads and is independently configured to provide a unique electronic circuit.

8. The apparatus according to claim 1, further comprising an electric current generator.

9. The apparatus according to claim 8, wherein the electric current generator is capable of producing an constant or variable electronic current.

10. The apparatus according to claim 9, wherein the variable electronic current varies in amplitude, duration, or frequency with respect to time.

11. The apparatus according to claim 9, wherein the variable electronic current varies according to a configurable predetermined program.

12. The apparatus according to claim 1, wherein the plurality of cell retaining units are optically transparent.

13. The apparatus according to claim 1, wherein the plurality of cell retaining units are constructed from glass or polycarbonate.

14. The apparatus according to claim 1, wherein the plurality of cell retaining units are electrically conductive.

15. The apparatus according to claim 1, wherein the plurality of cell retaining units have a surface that is at least partially coated with indium tin oxide.

16. The apparatus according to claim 1, wherein the first electrode and the second electrode are constructed from non-oxidative materials.

17. The apparatus according to claim 1, wherein the second electrode is at least partially constructed from gold, silver, copper, or an alloy thereof.

18. The apparatus according to claim 1, wherein the plurality of cell retaining units each comprise a surface upon which cells are capable of adhering.

19. The apparatus according to claim 1, wherein the cell retaining units are coated with a polymer that facilitates cellular adhesion.

20. The apparatus according to claim 19, wherein the polymer is polylysine.

21. The apparatus according to claim 1, wherein the plurality of cell retaining units is in the form of a multi-well plate.

22. A method of electronically stimulating cells comprising
providing a first electrode having a plurality of cell retaining units;
providing a second electrode having a plurality of electrically conductive leads complementary to the plurality of cell retaining units;
culturing cells within one or more of the cell retaining units, wherein the cells are adherent on a surface thereof and the cells are in an electrically conductive cell culture medium;
moving the first electrode into engagement with the second electrode to thereby complete a closed electronic circuit; and
applying an electronic current through the closed electronic circuit,
wherein all of the cells within each cell retaining unit are exposed to substantially the same electronic field.

23. The method according to claim 22, wherein the cell culture medium comprises an exogenous agent and the applied electronic current induces transient pores in the membranes of the cultured cells sufficient to permit the exogenous agent to enter the interior of at least of portion of the cultured cells.

24. The method according to claim 23, wherein the duration of the electric current is less than about 100 microseconds.

25. The method according to claim 23, wherein the exogenous agent comprises a polynucleotide.

26. The method according to claim 23, wherein the exogenous agent comprises a RNA molecule or DNA molecule.

27. The method according to claim 23, wherein the exogenous agent comprises a plasmid.

28. The method according to claim 23, wherein the exogenous agent comprises a siRNA molecule.

29. The method according to claim 23, wherein the exogenous agent comprises a polypeptide.

30. The method according to claim 23, wherein the exogenous agent comprises an antibody.

31. The method according to claim 23, wherein the exogenous agent causes a physiologic change in the cells.

32. The method according to claim 23, wherein the exogenous agent comprises a drug candidate.

33. The method according to claim 22, wherein the cultured cells comprise unicellular microorganisms.

34. The method according to claim 22, wherein the cultured cells comprise cells from a tissue of a multicellular organism.

35. The method according to claim 22, wherein the electronic current mimics the endogenous electronic stimulation encountered by the tissue in the multicellular organism.

36. The method according to claim 22, wherein the electronic is continuously applied.

37. A multi-well plate comprising
an optically transparent base having a surface coated with an optically transparent electric conductor; and
a plurality of non-electrically conducting walls attached to and extending vertically from the grid at their proximal ends,
wherein the plurality of walls and the base form a plurality of wells, each having an opening formed by the distal ends of the walls.

38. The multi-well plate according to claim 37, further comprising an electrically conductive strip applied to the perimeter walls, extending from the base to the distal ends of the perimeter walls.

39. The multi-well plate according to claim 37, further comprising an electrically conductive grid disposed between the walls and the base.

40. The multi-well plate according to claim 37, wherein the transparent electric conductor comprises a layer of indium tin oxide.

41. The multi-well plate according to claim 37, wherein the plurality of wells comprise 96 wells arranged in a 12 by 8 pattern, 384 wells arranged in a 12 by 24 pattern, or 1536 wells arranged in a 32 by 48 pattern.

42. An electrically conductive array comprising:
multiple electrodes complementary to multiple cell retaining units;
each of said multiple electrodes comprising a plurality of electrically conductive leads complementary to each cell retaining unit.

43. The array according to claim 42, further comprising a second electrode moveable into engagement with an electronically conductive cell culture medium to thereby provide a closed electronic circuit.

44. The array according to claim 42, whereby said plurality of electrically conductive leads can be inserted into a cell retaining unit simultaneously.

45. The array according to claim 42, whereby the distance of the plurality of electrode leads from the second electrode can be adjusted.

46. The array according to claim 42, whereby the plurality of electrode leads can be adjusted in three dimensions within each cell retaining unit.
